(19) United States
(12) Patent Application Publication
(10) Pub. No.: US 2025/0172524 A1
(43) Pub. Date: May 29, 2025

(54) AUTONOMOUS DIRECTIONAL MICROFLUIDIC DEVICES

(71) Applicant: President and Fellows of Harvard College, Cambridge, MA (US)

(72) Inventors: Mohamed Yafia Okba Salem, Cambridge, MA (US); Donald E. Ingber, Cambridge, MA (US); Adama Marie Sesay, Cambridge, MA (US); Jack Lindsay, Cambridge, MA (US); Hani M. Sallum, Cambridge, MA (US); Pawan Jolly, Cambridge, MA (US); Girija Goyal, Cambridge, MA (US)

(73) Assignee: President and Fellows of Harvard College, Cambridge, MA (US)

(21) Appl. No.: 18/832,921

(22) PCT Filed: Jan. 24, 2023
(86) PCT No.: PCT/US2023/011471

§ 371 Date: Jul. 24, 2024

Related U.S. Application Data
(60) Provisional application No. 63/385,254, filed on Nov. 29, 2022.
(60) Provisional application No. 63/407,508, filed on Sep. 16, 2022.
(60) Provisional application No. 63/302,856, filed on Jan. 25, 2022.

(51) Int. Cl. G01N 27/416; C12M 1/34; C12M 3/06; G01N 33/50

(52) U.S. Cl. CPC G01N 27/416; C12M 23/16; C12M 41/46; G01N 33/5082

(57) ABSTRACT

Devices, systems, and methods related to autonomous directional valves that allow fluids to stop and flow based on progressive changes in pressure are generally described.

### RELATED APPLICATIONS

This application claims priority under 35 U.S.C. § 119(e) to U.S. Provisional Patent Application No. 63/302,856, filed Jan. 25, 2022, U.S. Provisional Patent Application No. 63/407,508, filed Sep. 16, 2022, and U.S. Provisional Patent Application No. 63/385,254, filed Nov. 29, 2022, each of which are incorporated herein by reference in their entirety for all purposes.

### TECHNICAL FIELD

Devices, systems, and methods related to autonomous directional valves that allow fluids to stop and flow based on progressive changes in pressure are generally described.

### BACKGROUND

To perform controlled operations, conventional microfluidic devices employ valves requiring active mechanical or electromechanical elements, actuators, and/or controllers as well as external linked power sources. Such configurations limit the current microfluidic platforms from having a large number of valves that can enable numerous complex fluidic operations, and also increases the number of peripheral devices and production costs, therefore limiting the applicability of the devices. Moreover, applications like diagnostics and point of care testing require advanced automation with minimal peripherals.

### SUMMARY

Devices, systems, and methods related to autonomous directional valves that allow fluids to stop and flow based on progressive changes in pressure are generally described. The subject matter of the present disclosure involves, in some cases, interrelated products, alternative solutions to a particular problem, and/or a plurality of different uses of one or more systems and/or articles.

According to some embodiments, a microfluidic device is described, the microfluidic device comprising: a control channel; a first stop valve disposed along the control channel, wherein the first stop valve has a first burst pressure; a first reservoir and a second reservoir fluidly connected to the control channel at a position upstream from the first stop valve; a first flow path disposed between the control channel and the first reservoir, and a second flow path disposed between the control channel and the second reservoir, wherein the first flow path and the second flow path are separate from each other; and a second stop valve disposed between the control channel and the first reservoir, wherein the second stop valve has a second burst pressure, and wherein the second burst pressure is less than the first burst pressure.

In certain embodiments, a microfluidic device is described, the microfluidic device comprising a control channel and a plurality of cascading control reservoirs disposed in series along at least a portion of a length of the control channel, wherein each cascading control reservoir includes: a control reservoir with an inlet, wherein the inlet is fluidly connected to the control channel; a first stop valve disposed along the control channel, wherein the inlet of the control reservoir is positioned upstream from the first stop valve; a second stop valve disposed between the control channel and the inlet of the control reservoir; and a third stop valve fluidly connected to an outlet of the reservoir. The microfluidic device may, in some embodiments, comprise a plurality of process reservoirs, wherein each cascading control reservoir is fluidly coupled to one or more corresponding process reservoirs of the plurality of process reservoirs through the associated third stop valve, and wherein each cascading control reservoir is configured to apply a positive pressure to the one or more corresponding process reservoirs that causes a process fluid disposed in the one or more corresponding process reservoirs to flow.

According to certain embodiments, a method of flowing a process fluid in a microfluidic device is described, the method comprising: flowing a control fluid from a first portion of a control channel into a control reservoir when a pressure of the control fluid is greater than a first pressure threshold and below a second pressure threshold; flowing the process fluid in a process reservoir fluidly connected to the control reservoir; and flowing the control fluid from the first portion of the control channel to a second portion of the control channel when the pressure of the fluid is greater than the second pressure threshold.

In some embodiments, a microfluidic device is described, the microfluidic device comprising: a control channel; a first stop valve disposed along the control channel, wherein the first stop valve has a first burst pressure, and wherein the first stop valve extends out of a plane of the control channel; a first reservoir fluidly connected to the control channel at a position upstream from the first stop valve; and a second stop valve disposed between the control channel and the first reservoir, wherein the second stop valve has a second burst pressure, and wherein the second burst pressure is less than the first burst pressure.

According to certain embodiments, a microfluidic device is described, the microfluidic device comprising a control channel and a plurality of cascading control reservoirs disposed in series along at least a portion of a length of the control channel, wherein each cascading control reservoir includes: a control reservoir with an inlet, wherein the inlet is fluidly connected to the control channel; a first stop valve disposed along the control channel, wherein the inlet of the control reservoir is positioned upstream from the first stop valve, and wherein the first stop valve extends out of a plane of the control channel; a second stop valve disposed between the control channel and the inlet of the control reservoir; and a third stop valve fluidly connected to an outlet of the control reservoir. The microfluidic device may, in some embodiments, comprise a plurality of process reservoirs, wherein each cascading control reservoir is fluidly coupled to one or more corresponding process reservoirs of the plurality of process reservoirs through the associated third stop valve.

In some embodiments, a system is described, the system comprising: a microfluidic device; a reservoir pod; and an organ on chip, wherein the reservoir pod comprises a fluid reservoir, a fluid reservoir outlet, and an outlet port, wherein the fluid reservoir outlet is configured to be fluidly connected to a fluid inlet of the organ on chip, and wherein the fluid inlet of the microfluidic device is configured to be fluidly connected to a fluid outlet of the organ on chip through the outlet port of the reservoir pod.

According to certain embodiments, a method of flowing a fluid through a system is described, the method comprising flowing the fluid from a fluid reservoir of a reservoir pod into an organ on chip, and flowing the fluid through the organ on chip to a microfluidic device.

Other advantages and novel features of the present disclosure will become apparent from the following detailed description of various non-limiting embodiments when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand. In the figures:

FIG. 1 shows, according to some embodiments, a schematic diagram of a microfluidic device comprising a control channel and a reservoir;

FIG. 2A-2D show, according to some embodiments, a method of fabricating a microfluidic device;

FIG. 3 shows, according to some embodiments, a schematic diagram of a microfluidic device comprising a plurality of reservoirs having an inlet positioned upstream form a first stop valve;

FIG. 4 shows, according to some embodiments, a schematic diagram of a microfluidic device comprising a control channel, a control reservoir, and a process reservoir;

FIG. 5 shows, according to some embodiments, a schematic diagram of a microfluidic device comprising a control channel and a plurality of cascading reservoirs;

FIG. 6 shows, according to some embodiments, a schematic diagram of a microfluidic device comprising a control channel, a plurality of cascading control reservoirs, and a plurality of process reservoirs;

FIG. 7A shows, according to some embodiments, a schematic diagram of a microfluidic device comprising a control channel and a reservoir, depicting a method of flowing a fluid through the microfluidic device;

FIG. 7B shows, according to some embodiments, a schematic diagram of a microfluidic device comprising a control channel, a control reservoir, and a process reservoir, depicting a method of flowing a control fluid and a process fluid through the microfluidic device;

FIG. 8A shows, according to some embodiments, a schematic diagram of a microfluidic device comprising a plurality of passive components that extend out of a plane relative to an adjacent component that each passive component is fluidly connected to;

FIG. 8B shows, according to some embodiments, a schematic diagram of an inlet reservoir, a control channel, a first stop valve, a second stop valve, and a control reservoir of the microfluidic device shown in FIG. 8A, wherein the inlet reservoir, the first stop valve, and the control reservoir extend out of a plane relative to an adjacent component that each passive component is fluidly connected to;

FIG. 8C shows, according to some embodiments, a schematic diagram of a control reservoir, an outlet, and a third stop valve of the microfluidic device shown in FIG. 8A, wherein the control reservoir and the third stop valve extend out of a plane relative to an adjacent component that each passive component is fluidly connected to;

FIG. 8D shows, according to some embodiments, a schematic diagram of a process reservoir and an outlet channel of the microfluidic device shown in FIG. 8A, wherein the process reservoir extends out of a plane relative to an adjacent component that the passive component is fluidly connected to;

FIG. 9 shows, according to some embodiments, a perspective view schematic diagram of the first stop valve shown in FIG. 8B;

FIG. 10A shows, according to some embodiments, a top-view schematic diagram of a reservoir pod;

FIG. 10B shows, according to some embodiments, a perspective view schematic diagram of a reservoir pod;

FIG. 10C shows, according to some embodiments, a cross-sectional schematic diagram of an outlet port taken along line 10C in FIG. 10A;

FIG. 11A shows, according to some embodiments, a schematic diagram of a microfluidic device comprising a fluid inlet, a control channel, and a reservoir;

FIG. 11B shows, according to some embodiments, a schematic diagram of a microfluidic device comprising a fluid inlet, a control channel, and a plurality of reservoirs;

FIG. 12 shows, according to some embodiments, an exploded view schematic diagram of a microfluidic device and a reservoir pod;

FIG. 13A shows, according to some embodiments, a top-view schematic diagram of a microfluidic device and a connected reservoir pod;

FIG. 13B shows, according to some embodiments, a cross-sectional schematic diagram of a microfluidic device and a connected reservoir pod taken along line 13B in FIG. 13A;

FIG. 13C shows, according to some embodiments, a cross-sectional schematic diagram of a microfluidic device and a connected reservoir pod taken along circle 13C in FIG. 13B;

FIG. 14A shows, according to some embodiments, a top-view schematic diagram of an organ on chip;

FIG. 14B shows, according to some embodiments, cross-sectional schematic diagram of an organ on chip;

FIG. 15 shows, according to some embodiments, an embodiment of a system comprising a microfluidic device, a reservoir pod, and an organ on chip;

FIG. 16 shows, according to some embodiments, a process flow diagram depicting a method of a flowing a fluid in a system;

FIG. 17A shows, according to some embodiments, a calibration curve for detection of MIP-1β;

FIG. 17B shows, according to some embodiments, a calibration curve for detection of TIMP-1;

FIG. 18A shows, according to some embodiments, the peak current height of 100 pg/mL of MIP-1β and 650 pg/mL of TIMP-1 as a function of incubation time from 1 to 19 hours;

FIG. 18B shows, according to some embodiments, the peak current height of 650 pg/mL of MIP-1β and 100 pg/mL of TIMP-1 as a function of incubation time from 1 to 19 hours;

FIG. 19A shows, according to some embodiments, the detection of TIMP-1 without cross-reactivity;

FIG. 19B shows, according to some embodiments, the detection of MIP-1β without cross-reactivity; and

FIG. 20 shows, according to some embodiments, the detection of MIP-1β and TIMP-1 without cross reactivity.

### DETAILED DESCRIPTION

Microfluidics systems that are used to enable miniaturization of devices and instruments require advanced levels of automation and active control elements to execute sequential complex operations needed for performing chemical and biological assays. Microfluidic valves are the main components needed to automate microfluidic operations. The valves are used to stop fluid flow and restart the flow again in a timed stepped manner, which helps in performing complex microfluidic operations such as moving defined volumes of samples and/or reagents to different locations of the microfluidic system and performing incubation, mixing, and/or sample analysis at defined time points.

Commonly used conventional valves require mechanical moving parts to control the fluid flow, which can be actuated manually or using solenoid actuators that need multiple electrical connections, controllers, and/or power sources. Other conventional valves, such as pneumatic valves, can control fluid flow by applying positive pressure on an elastic pneumatic channel that can block flow in a hydraulic channel. These valves can automate many operations, but require a larger number of pneumatic connections and external peripherals. Mechanical microvalves use internal moving structures to control fluid flow, but require connections to external pressure sources to control the internal moving structures inside each valve. Electro-actuated valves have been introduced to microfluidic systems to enable liquid flow without pumps, but the electro-actuated valves still require external electrical connections. In summary, the aforementioned valves used in conventional microfluidic devices require mechanical moving parts, controllers, pressure sources, and/or power sources to facilitate fluid flow.

Domino capillary microfluidic systems have been developed to enable sequential liquid delivery without external peripherals, however the domino system cannot enable autonomous sequential liquid sampling steps needed for storing liquid effluent(s) of different biological or other desirable chemical/fluid processes. Moreover, the domino system requires precise surface treatments and constructions. The domino system also requires linking all reservoirs with a main drainage channel connected to a pump so that sequential liquid operations can be performed.

The Inventors have realized and appreciated that microfluidic devices comprising passive directional valves can be configured to automate complex microfluidic operations without any external peripherals and/or electrical connections. A microfluidic device may have a control channel, a first stop valve disposed along the control channel, a reservoir fluidly connected to the control channel at a position upstream from the first stop valve, and a second stop valve disposed between the control channel and the reservoir. The stop valves may be configured such that the first stop valve has a first burst pressure and the second stop valve has a second burst pressure, wherein the second burst pressure is less than the first burst pressure. Configuring the device in this way allows fluids (e.g., liquids) to stop and flow in a programmed manner based on the progressive changes in pressure that result from the design of the stop valves. A fluid flowing in the control channel, for example, may have a pressure that is greater than a first pressure threshold corresponding to the burst pressure of the second valve (e.g., the second burst pressure), but less than a second pressure threshold corresponding to the burst pressure of the first stop valve (e.g., the first burst pressure). The pressure of the fluid may thereby cause the second stop valve to burst without bursting the first valve. The fluid may sequentially flow into the reservoir, thereby optionally filling the reservoir and increasing the pressure of the fluid until the pressure is greater than the second pressure threshold corresponding to the burst pressure of the first stop valve (e.g., the first burst pressure), which causes the fluid to burst the first stop valve. A third stop valve with a burst pressure greater than both the first stop valve and the second valve may be disposed at an outlet of the reservoir, which causes the fluid flow to stop in the reservoir and redirects the flow towards the first stop valve.

The design of two valves with varying burst pressures as explained above may be employed such that a microfluidic device includes a plurality of cascading reservoirs disposed in series along a portion of a length of a control channel. In some embodiments, for example, each cascading reservoir may include a reservoir fluidly connected to the control channel, a first stop valve configured such that an inlet of the reservoir is positioned upstream from the first stop valve, and a second stop valve disposed between the control channel and the reservoir inlet. Each reservoir may also include a third stop valve disposed at the outlet of each reservoir that inhibits fluid flow. The implementation of the autonomous directional valves in the cascaded configuration allows fluids (e.g., liquids) to stop and flow in a programmed manner based on progressive changes in pressures that result from the arrangement of valves in the microfluidic structure. According to some embodiments, for example, when the pressure of the fluid in the reservoir (e.g., the first reservoir) causes the fluid to burst the stop valve disposed in the control channel upstream from the reservoir, as explained above, the fluid continues flowing in the control channel and enters the next cascading reservoir positioned in series. Upon entering the next cascading reservoir, the fluid may burst the stop valve disposed between the control channel and the reservoir inlet, optionally fill the reservoir, and subsequently burst the stop valve upstream from the reservoir. The microfluidic device may be constructed such that the number of cascading reservoirs is suitable for the specific application of the device.

The microfluidic device may be configured such that each of the plurality of cascading reservoirs are control reservoirs that are fluidly connected to a process reservoir. In certain embodiments, for example, the flow of fluid passing through the cascading control reservoirs applies a positive pressure that can move and displace liquid samples and/or reagents stored in the process reservoirs. The stop valve disposed at the outlet of each control reservoir stops the liquid in the control reservoirs from entering the process reservoirs, but allows a positive pressure applied from the liquid flowing in the control reservoirs to flow the process fluid disposed in the process reservoir.

While any appropriate construction may be used, including, for example, microfluidic devices made with typical additive and/or subtractive manufacturing techniques (e.g., 3D-printing), in some embodiments, the microfluidic devices described herein may be fabricated using one or more patterned films wherein each film has at least a portion of a profile of a desired portion of the microfluidic device (e.g., a channel, a reservoir, a stop valve, other microfluidic components) formed in the one or more patterned films. The one or more patterned films may be stacked together (e.g., bonded) to form the microfluidic device. In certain embodiments, for example, the device may comprise a substrate, one or more patterned intermediate layers that define a plurality of passive elements (e.g., channels, valves, reservoirs, other microfluidic components), and a top layer to cover the microfluidic device. In some embodiments, certain portions of the device may be rendered hydrophilic or hydrophobic depending on the material used to fabricate the device, thereby assisting or inhibiting fluid flow at specific locations within the device.

The pressure difference between each of the stop valves may be provided by the design of the microfluidic device since the pressure can be controlled by the stop valve geometry and surface chemistry. In some embodiments, for example, the burst pressure of the stop valves can be controlled by adjusting the dimensions of the channel and/or varying the design between stepped and ramped configurations, wherein the burst pressure of the stop valve is inversely proportional with the stop valve dimension.

According to certain embodiments, regardless of the construction technique used to fabricate the microfluidic device, one or more passive components (e.g., channels, valves, reservoirs, other microfluidic components) may extend out of a plane relative to an adjacent component that the one or more passive components is fluidly connected to. In some embodiments, for example, a microfluidic device comprises one or more inlet reservoirs, control channels, stop valves, control reservoirs, process reservoirs, and/or outlet channels that extend out of a plane relative to an adjacent component that the one or more inlet reservoirs, control channels, stop valves, control reservoirs, process reservoirs, and/or outlet channels is fluidly connected to. As described above, for example, a microfluidic device may comprise a control channel and a stop valve disposed along the control channel, wherein at least one dimension of the stop valve, such as a depth of the stop valve, may extend out of a plane relative to the control channel (e.g., an upstream portion of the control channel and/or a downstream portion of the control channel). In some embodiments, such a configuration advantageously increases the resistance of a fluid disposed in the stop valve from leaking across the stop valve.

In certain embodiments, at least one dimension of a passive component may be larger than a corresponding dimension of an adjacent component that the passive component is fluidly connected to. In some embodiments wherein a microfluidic device comprises a control channel and a stop valve disposed along the control channel, for example, at least one dimension (e.g., a width) of the stop valve may be larger than a corresponding dimension (e.g., a width) of the control channel (e.g., an upstream portion of the control channel and/or a downstream portion of the control channel). Such a configuration advantageously increases the resistance of a fluid disposed in the stop valve from leaking across the stop valve, according to some embodiments.

According to some embodiments, passive components that extend out of a plane relative to an adjacent component that the passive component is fluidly connected to may be capable of handling complex fluids, samples, and/or reagents (even those with low surface tensions) without, for example, unintentionally bursting and/or leaking through the passive component. The passive components that extend out of a plane relative to an adjacent component that the passive component is fluidly connected to stand in contrast to conventional microfluidic devices comprising planar (e.g., two dimensional) passive elements, such as planar stop valves, which fail, in some cases, under positive pressure. In some cases, for example, conventional planar stop valves may be fabricated in a microfluidic channel by altering one or more two-dimensional geometric dimensions of the channel, such as a width of the channel and/or an angle of a diverging outlet of the channel. Such conventional planar stop valves may be prone to leakage due to fluids (e.g., low surface tension fluids) wicking into one or more corners and/or edges of the planar stop valve, thereby causing the planar stop valve to burst unintentionally. In contrast, a passive component that extends out of a plane relative to an adjacent component that the passive component is fluidly connected to, as described herein, allows for casier control of fluids (e.g., low surface tensions fluids) due to the fluid having a higher resistance to flow at the dimension that extends out of the plane, thereby eliminating issues associated with leakage and/or unintentional bursting of stop valves.

The microfluidic device can be used for performing various types of advanced operations without any additional peripherals, connections, and/or controllers, thereby allowing discrete, simultaneous, and sequential operations. The devices described herein can enable complex operations to be carried out with the device, thereby providing true lab-on-a-chip applications that can, for example, process logic gates encoded in the design of the microfluidic device, such as AND, OR, NOT gates that represent the basic building blocks of microprocessors, without requiring any surface treatment or mechanical or electromechanical actuators, interfaces, and/or external power sources.

According to some embodiments, the microfluidic devices disclosed herein may be implemented in a system comprising a reservoir pod and an organ on chip (e.g., a human lymphoid follicle-on-chip). The system may include a number of fluidic connections between the microfluidic device, the reservoir pod, and the organ on chip such that the microfluidic device is configured to test an output (e.g., an effluent) of the organ on chip. In certain embodiments, for example, the microfluidic device may be configured to perform in-line continuous and multiplexed monitoring of an output of an organ on chip that replicates a biological environment for monitoring conditions (e.g., diseased conditions) and/or responses (e.g., vaccination responses) overtime. For example, the microfluidic device may, in some embodiments, include one or more sensors that are configured to detect one or more analytes, such as one or more biomarkers (e.g., cytokines), that may be present in the output of the organ on chip. In certain embodiments, an effluent fluid sample and/or culture medium from the organ on chip may be drawn into the microfluidic device at regular intervals such that each analyte of the one or more analytes is separately detected.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

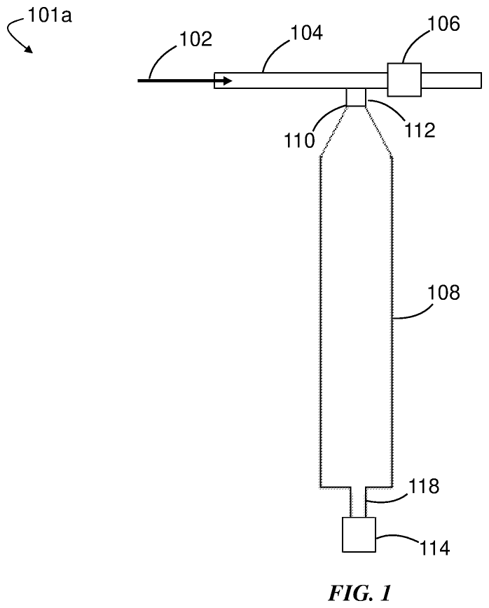

In some embodiments, a microfluidic device comprises a control channel. FIG. 1 shows, according to some embodiments, a schematic diagram of microfluidic device 101a comprising control channel 104.

Although FIG. 1 shows that control channel 104 is configured in a straight direction, the control channel may have other directional configurations, as the disclosure is not meant to be limiting in this regard. In some embodiments, for example, the control channel may be configured with any of a variety of directional turns (e.g., 45° angles, 90° angles, etc.) and/or curves.

According to certain embodiments, a microfluidic device comprises a first stop valve disposed along the control channel. Referring, for example, to FIG. 1, microfluidic device 101a comprises first stop valve 106 disposed along control channel 104.

In certain embodiments, the first stop valve may have a first burst pressure. The first burst pressure may be any of a variety of suitable burst pressures. In some embodiments, the burst pressure of the first stop valve is greater than or equal to 200 Pa, greater than or equal to 300 Pa, greater than or equal to 400 Pa, greater than or equal to 500 Pa, or greater than or equal to 600 Pa. In certain embodiments, the burst pressure of the first stop valve is less than or equal to 700 Pa, less than or equal to 600 Pa, less than or equal to 500 Pa, less than or equal to 400 Pa, or less than or equal to 300 Pa. Combinations of the above recited ranges are possible (e.g., the burst pressure of the first stop valve is between greater than or equal to 200 Pa and less than or equal to 700 Pa, the burst pressure of the first stop valve is between greater than or equal to 400 Pa and less than or equal to 500 Pa). Other ranges are also possible. It should be noted that pressures other than the pressures listed above are also possible, as the disclose is not meant to be limiting in this regard.

In certain embodiments, a microfluidic device comprises a reservoir (e.g., a first reservoir) fluidly connected to the control channel. For example, FIG. 1 shows microfluidic device 101a comprising reservoir 108 fluidly connected to control channel 104. The reservoir may be fluidly connected to the control channel at a position upstream from the first stop valve. Referring to FIG. 1, for example, reservoir 108 comprises inlet 110 fluidly connected to control channel 104 and positioned upstream from first stop valve 106.

According to some embodiments, a microfluidic device comprises a second stop valve disposed between the control channel and the reservoir. As shown in FIG. 1, for example, microfluidic device 101a comprises second stop valve 112 disposed between control channel 104 and reservoir 108. The second stop valve may, in some embodiments, be positioned at a fluidic interface between the control channel and the reservoir. For example, referring to FIG. 1, inlet 110 of reservoir 108 is fluidly connected to second stop valve 112.

In certain embodiments, the second stop valve has a second burst pressure. The second burst pressure may be less than the first burst pressure (e.g., the burst pressure of the first stop valve), according to some embodiments. Referring, for example, to FIG. 1, the burst pressure of second stop valve 112 (e.g., the second burst pressure) may be less than the burst pressure of first stop valve 106 (e.g., the first burst pressure). Configuring the microfluidic device in this way advantageously allows a fluid to burst the second stop valve positioned at the fluidic interface between the control channel and the reservoir and autonomously flow into (and optionally fill) the reservoir without bursting the first stop valve. The resistance at the second stop valve may be lower than the resistance at the first stop valve, according to some embodiments.

The second burst pressure may be any of a variety of suitable pressures, provided that the second burst pressure is less than the first burst pressure. In some embodiments, the burst pressure of the second stop valve is greater than or equal to 50 Pa, greater than or equal to 75 Pa, greater than or equal to 100 Pa, greater than or equal to 125 Pa, greater than or equal to 150 Pa, or greater than or equal to 175 Pa. In certain embodiments, the burst pressure of the second stop valve is less than or equal to 200 Pa, less than or equal to 175 Pa, less than or equal to 150 Pa, less than or equal to 125 Pa, less than or equal to 100 Pa, or less than or equal to 75 Pa. Combinations of the above recited ranges are possible (e.g., the burst pressure of the second stop valve is between greater than or equal to 50 Pa and less than or equal to 200 Pa, the burst pressure of the second stop valve is between greater than or equal to 100 Pa and less than or equal to 125 Pa). Other ranges are also possible. It should be noted that pressures other than the pressures listed above are also possible, as the disclose is not meant to be limiting in this regard.

According to some embodiments, a microfluidic device may comprise a third stop valve fluidly connected to an outlet of the reservoir. Referring, for example, to FIG. 1, microfluidic device 101a comprises third stop valve 114 fluidly connected to outlet 118 of reservoir 108. In some embodiments, and as shown in FIG. 1, third stop valve 114 is fluidly connected to a downstream portion (e.g., a most downstream portion) of reservoir 108.

In some embodiments, the third stop valve has a third burst pressure. According to some embodiments, the first burst pressure (e.g., the burst pressure of the first stop valve) may be less than the third burst pressure. Referring to FIG. 1, for example, the burst pressure of first stop valve 106 (e.g., the first burst pressure) may be less than the burst pressure of third stop valve 114 (e.g., the third burst pressure). Configuring the microfluidic device in this way advantageously allows a fluid to burst the first stop valve disposed along the control channel without bursting the third stop valve.

The third burst pressure may be any of a variety of suitable pressures, provided that the third burst pressure is greater than the first burst pressure. In some embodiments, the burst pressure of the third stop valve is greater than or equal to 700 Pa, greater than or equal to 800 Pa, greater than or equal to 900 Pa, greater than or equal to 1000 Pa, greater than or equal to 1200 Pa, greater than or equal to 1400 Pa, greater than or equal to 1600 Pa, or greater than or equal to 1800 Pa. In certain embodiments, the burst pressure of the third stop valve is less than or equal to 2000 Pa, less than or equal to 1800 Pa, less than or equal to 1600 Pa, less than or equal to 1400 Pa, less than or equal to 1200 Pa, less than or equal to 1000 Pa, less than or equal to 900 Pa, or less than or equal to 800 Pa. Combinations of the above recited ranges are possible (e.g., the burst pressure of the third stop valve is between greater than or equal to 700 Pa and less than or equal to 2000 Pa, the burst pressure of the second stop valve is between greater than or equal to 1000 Pa and less than or equal to 1200 Pa). Other ranges are also possible. It should be noted that pressures other than the pressures listed above are also possible, as the disclose is not meant to be limiting in this regard.

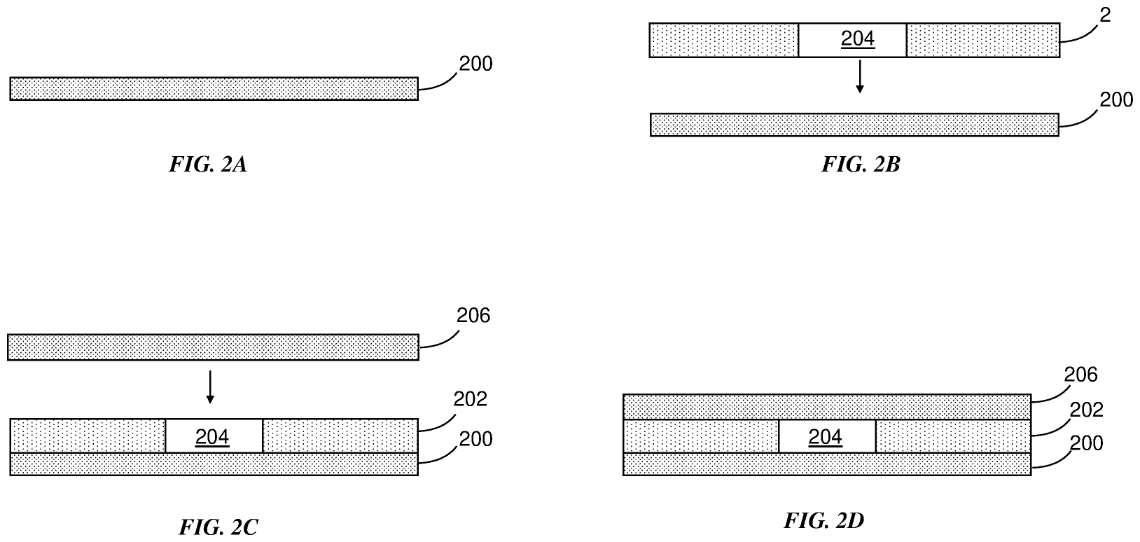

The microfluidic devices described herein may be fabricated by any of a variety of suitable means. In some embodiments, the microfluidic devices are fabricated using individual layers to form different portions of the microfluidic components. Such a fabrication method advantageously enables the use of different materials for various portions of a microfluidic device and facilitates the fabrication of devices with tailored geometries and/or surface properties. FIGS. 2A-2D show, according to some embodiments, a cross-sectional schematic diagram depicting a method of fabricating a microfluidic device. According to some embodiments, for example, a microfluidic device may comprise substrate 200, one or more intermediate layers 202, and top layer 206. In certain embodiments, one or more intermediate layers 202 may be configured to at least partially define a plurality of fluidly connected components (e.g., channels, valves, reservoirs, other microfluidic components). In some embodiments, for example, one or more intermediate layers 202 may be patterned such that one or more intermediate layers 202 comprise one or more features 204 corresponding to a passive component of the microfluidic device (e.g., channels, valves, reservoirs, other microfluidic components). In some embodiments, the feature may be, for example, a control channel, a stop valve (e.g., a first stop valve, a second stop valve, a third stop valve, etc.), a reservoir (e.g., a control reservoir, a process reservoir, etc.). Other fluidly connected components are also possible.

Depending on the function of the feature (e.g., channel, valve, reservoir, other microfluidic component) formed in the one or more intermediate layers, the overall size, shape, and/or geometry of the feature may be tailored for a particular application. Such a configuration advantageously enables the fabrication of microfluidic devices that can be fine-tuned to perform advanced microfluidic operations, including sequential delivery and/or mixing of liquid samples and/or reagents, delivery and/or mixing of defined volumes of liquid samples and/or reagents, timed and/or stepped operations using sacrificial reservoirs that can act as timers, stopping and incubating liquid samples, and starting and stopping flow through a device using sacrificial reservoirs, by precisely controlling the capillary pressures at desired locations within the device. For example, although not shown in the figures, a microfluidic device may comprise more than one intermediate layer (e.g., two intermediate layers, three intermediate layers, four intermediate layers, etc.), and one or more features may be formed in more than one intermediate layer. For example, in some embodiments, one or more features with a desired large geometry (e.g., cross-sectional dimension), such as a reservoir, channel, and/or stop valve with a low burst pressure, may be formed in more than one intermediate layer. In certain other embodiments, one or more features with a desired small geometry, such as a channel and/or stop valve with a large burst pressure, may be formed in one intermediate layer.

In some embodiments, one or more intermediate layers of the microfluidic device are manufactured with a set of corresponding patterns where portions of material have been removed. At least portions of the patterns of the individual layers align with one another when stacked together such that the layers define a plurality of interconnected channels, reservoirs, valves, and/or other microfluidic components. The patterns of removed material in the intermediate layers may be formed, in some embodiments, by cutting, stamping, punching, and/or etching the patterns into the one or more intermediate layers. In certain non-limiting embodiments, for example, the patterns corresponding to the plurality of channels, reservoirs, valves, and/or other microfluidic components may be cut into the one or more intermediate layers using plot cutters and/or laser cutters, such as a Silhouette portrait craft cutter (Silhouette America, Lindon, UT) and/or a Graphtec Cutting Plotter CE-5000 (Graphtec America, Inc., Irvine, CA). Of course, any other appropriate method of forming a pattern in the one or more intermediate layers may also be used. In certain embodiments, for example, the patterns corresponding to the plurality of channels, reservoirs, valves, and/or other microfluidic components are formed in the one or more intermediate layers by additive manufacturing (e.g., 3D-printing). Different fabrication methods (e.g., cutting, stamping, punching, etching, additive manufacturing, etc.) of the one or more intermediate layers can affect the surface properties of the layer.

After forming the desired patterns in the one or more intermediate layers, the method may include applying the one or more intermediate layers to a substrate. Referring, for example, to FIGS. 2A and 2B, intermediate layer 202 comprising feature 204 is positioned over substrate 200 in a desired position and orientation prior to placing intermediate layer 202 in contact with substrate 200 such that intermediate layer 202 is disposed on substrate 200. As explained herein, feature 204 may correspond to a channel, reservoir, valve, and/or other microfluidic components. While not depicted, in some embodiments, one or more additional intermediate layers may also be positioned over and placed into contact with the first intermediate layer in a desired position and orientation such that they are disposed on the first intermediate layer (e.g., on a surface of the first intermediate layer opposite from the substrate). Thus, complex microfluidic structures defined by interconnected patterns formed in multiple intermediate layers may be constructed. As shown in FIG. 2C, in certain embodiments, the method also includes positioning top layer 206 over a topmost one of one or more intermediate layers 202 in a desired position and orientation. Top layer 206 may then be placed into contact with the topmost one of one or more intermediate layers 202 such that one or more intermediate layers 202 are disposed between top layer 206 and substrate 202, as shown in FIG. 2D. Once assembled, the various microfluidic components such as channels, valves, reservoirs, and/or other microfluidic components may be formed between the surfaces of the stacked substrate, one or more intermediate layers, and top layer.

In some embodiments, the microfluidic devices described herein may be fabricated using methods described in U.S. Patent Application No. 63/047,012, filed Jul. 1, 2020, and entitled “Microfluidic Systems Containing Layers of Films,” and in U.S. Patent Application No. 63/302,832, filed Jan. 25, 2022, and entitled “Microfluidic Systems Containing Layers of Films,” which are incorporated herein by reference in their entirety.

According to certain embodiments, at least a portion of a microfluidic device as described herein may be fabricated by additive manufacturing (e.g., 3D-printing). Any of a variety of suitable additive manufacturing processes may be employed. In certain embodiments, for example, suitable additive manufacturing processes include fused deposition modeling (FDM), fused filament fabrication (FFF), stercolithography (SLA), and/or selective laser sintering (SLS), although other additive manufacturing processes may also be employed, as the disclosure is not meant to be limiting in this regard.

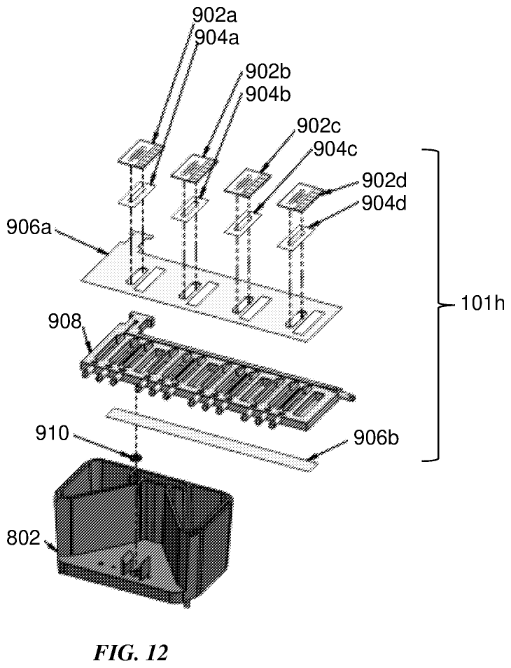

FIG. 12 shows, according to some embodiments, an exploded view schematic diagram of a microfluidic device. The microfluidic device may, in some embodiments, comprise microfluidic device body 908. In certain embodiments, microfluidic device body 908 comprises any of the components (e.g., passive components) described herein, including, for example, a control channel, a first stop valve (e.g., having a first burst pressure) disposed along the control channel, a reservoir fluidly connected to the control channel, a second stop valve (e.g., having a second burst pressure) disposed between the control channel and the reservoir, a third stop valve (e.g., having a third burst pressure), and/or any other appropriate arrangement of the various components disclosed herein that may be fluidly connected to an outlet of the reservoir. Of course, other microfluidic devices may be used with the disclosed systems as the disclosure is not so limited.

In certain embodiments, the microfluidic device body and/or the components (e.g., passive components) thereof may be fabricated by additive manufacturing (e.g., 3D-printing). In certain embodiments, for example, one or more features corresponding to a component (e.g., passive component) of the microfluidic device (e.g., channels, valves, reservoirs, other microfluidic components) may be formed in the microfluidic device body by additive manufacturing.

Any of a variety of suitable additive manufacturing materials may be employed to fabricate the microfluidic device body and/or the components thereof. According to some embodiments, the additive manufacturing material may be chosen by a person of ordinary skill in the art to avoid corrosion and/or deterioration of the microfluidic device and/or components (e.g., passive components) thereof caused by, for example, temperature and/or pressure fluctuations within the microfluidic device and/or from the fluids flowing therein. In certain embodiments, the additive manufacturing material comprises a thermoplastic, a thermosetting plastic, and/or a curable resin. The additive manufacturing material may, in some embodiments, comprise acrylonitrile butadiene styrene (ABS), a thermoplastic elastomer (TPE), a thermoplastic polyurethane (TPU), polylactic acid (PLA), high impact polystyrene (HIPS), polyethylene terephthalate (PET), glycol modified polyethylene terephthalate (PETG), nylon (e.g., polyamide), acrylic styrene acrylonitrile (ASA), polycarbonate, polypropylene, polyvinyl alcohol, cyclo olefin polymer (COP), cyclic olefin copolymer (COC), carbon fiber filaments, metal filaments, and/or wood filaments. Other additive manufacturing materials are also possible.

According to certain embodiments, at least a portion of a microfluidic device as described herein may be fabricated by injection molding, hot embossing, and/or laser cutting. In some embodiments, for example, the microfluidic device, the microfluidic device body, and/or the components (e.g., passive components) thereof may be fabricated by injection molding, hot embossing, and/or laser cutting.

Advantageously, the microfluidic devices described herein (e.g., fabricated using individual layers to form different portions of the microfluidic components, fabricated using additive manufacturing) are capable of direct use (e.g., in assays) with the native contact angles of the materials (e.g., polymers, plastics) used to fabricate the device, without the need for any hydrophilic and/or hydrophobic surface treatment. Furthermore, the microfluidic devices are robust and capable of handling a wide array of liquids, samples, and reagents, even those with low surface tensions.

As explained above, a microfluidic device as described herein may be configured with a number of stop valves having various burst pressures. In some embodiments, the burst pressure of the stop valves can be controlled by adjusting the dimensions of the stop valve. For example, in some embodiments, the dimensions of one or more features in the one or more intermediate layers corresponding to a stop valve may be adjusted in order to tune the burst pressure of the stop valve. In other embodiments, the dimensions of one or more features corresponding to a stop valve formed by additive manufacturing may adjusted in order to tune the burst pressure of the stop valve. According to some embodiments, the burst pressure of the stop valve can be controlled by employing a stop valve that extends out of a plane relative to an adjacent component that the stop valve is fluidly connected to, as explained herein in greater detail. In some embodiments, the burst pressure of the stop valve can be controlled by employing a stop valve having at least dimension that is larger than a corresponding dimension of an adjacent component that the stop valve is fluidly connected to, as explained herein in greater detail. In certain embodiments, the burst pressure of the stop valve is inversely proportional to the stop valve dimension, such that a stop valve with a smaller geometry requires a higher burst pressure to burst the stop valve as compared to a stop valve with a larger geometry.

In certain embodiments, regardless of the fabrication technique, the burst pressure of the stop valves can be controlled by varying the design of the stop valve between stepped and ramped configurations. In certain embodiments, for example, the intermediate layers of the microfluidic device may be configured such that there is a feature patterned into the intermediate layers comprising a stepped configuration between intermediate layers, thereby functioning as a stop valve due to the step change in height. In some embodiments, the feature formed by additive manufacturing may comprise a stepped configuration, thereby functioning as a stop valve due to the step change in height. In other embodiments, the feature patterned into the intermediate layers may comprise a ramped configuration between intermediate layers, thereby functioning as a stop valve due to the gradual change in height. In certain other embodiments, the feature formed by additive manufacturing may comprise a ramped configuration, thereby functioning as a stop valve due to the gradual change in height.

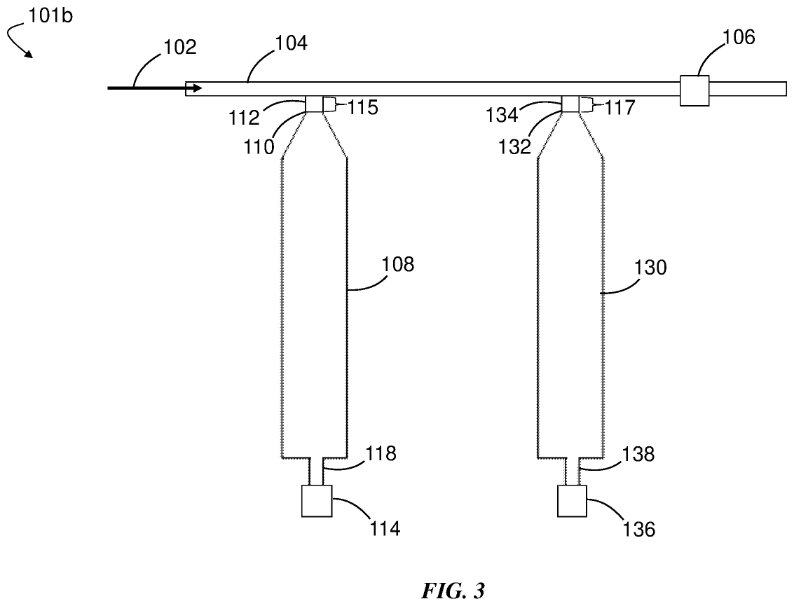

According to some embodiments, a microfluidic device may comprise more than one reservoir fluidly connected to the control channel at a position upstream from the first stop valve such that flow into the multiple reservoirs upstream from the first stop valve may be controlled by the state of the first stop valve. FIG. 3 shows, according to some embodiments. a schematic diagram of a microfluidic device comprising a plurality of reservoirs having an inlet positioned upstream from a first stop valve. Referring to FIG. 3, microfluidic device 101b may, in some embodiments, comprise reservoir 108 (e.g., first reservoir) and reservoir 130 (e.g., second reservoir). In some embodiments, first reservoir 108 and second reservoir 130 are fluidly connected to control channel 104 at a position upstream from first stop valve 106. For example, in certain embodiments, first reservoir comprises inlet 110 positioned upstream from first stop valve 106 and second reservoir 130 comprises inlet 132 positioned upstream from first stop valve 106.

According to some embodiments, the microfluidic device may comprise a first flow path disposed between the control channel and the first reservoir. Referring, for example, to FIG. 3, microfluidic device 101b comprises first flow path 115 disposed between control channel 104 and first reservoir 108. First flow path 115 may, in some embodiments. comprise a flow path through second stop valve 112, as shown in FIG. 3. In some embodiments, although not shown in the figures, the first flow path may comprise a flow path through one or more passive components (e.g., a channel) in addition to the second stop valve, as the disclosure is not meant to be limiting in this regard.

The second reservoir may include a stop valve disposed between the control channel and the second reservoir. Referring, for example, to FIG. 3, microfluidic device 101b comprises fourth stop valve 134 disposed between control channel 104 and second reservoir 130. Fourth stop valve 134 is, in some embodiments, positioned at a fluidic interface between control channel 104 and second reservoir 130 (e.g., inlet 132 of second reservoir 130 is fluidly connected to fourth stop valve 134).

The fourth stop valve may have a fourth burst pressure. In some embodiments, the second burst pressure (e.g., the burst pressure of the second stop valve) is less than the fourth burst pressure. Referring, for example, to FIG. 3, the burst pressure of second stop valve 112 (e.g., the second burst pressure) may be less than the burst pressure of fourth stop valve 134 (e.g., the fourth burst pressure). Configuring the microfluidic device in this way allows a fluid to flow into (and optionally fill) the first reservoir prior to flowing into the second reservoir. In certain embodiments, the fourth burst pressure is less than the first burst pressure (e.g., the burst pressure of the first stop valve). For example, referring to FIG. 3, the burst pressure of fourth stop valve 134 (e.g., the fourth burst pressure) is less than the burst pressure of first stop valve 106 (e.g., the first burst pressure). The microfluidic device may be advantageously configured in this way so that a fluid can burst the fourth stop valve positioned at the fluidic interface between the control channel and the second reservoir and autonomously flow into (and optionally fill) the second reservoir without bursting the first stop valve.

The fourth burst pressure may be any of a variety of suitable pressures described above with respect to the second burst pressure, provided that the second burst pressure is less than the fourth burst pressure and the fourth burst pressure is less than the first burst pressure.

According to certain embodiments, the microfluidic device may be configured such that the second stop valve and the fourth stop valve are configured to burst simultaneously. In some embodiments, for example, the second burst pressure (e.g., the burst pressure of the second stop valve is the same as the fourth burst pressure (e.g., the burst pressure of the fourth stop valve). Referring, for example, to FIG. 3, the burst pressure of second stop valve 112 (e.g., the second burst pressure) may be the same as the burst pressure of fourth stop valve 134 (e.g., the fourth burst pressure). Configuring the microfluidic device in this way allows a fluid to flow into (and optionally fill) the first reservoir while the fluid flows into (and optionally fills) the second reservoir. In some such embodiments wherein the second stop valve and the fourth stop valve are configured to burst simultaneously, the second burst pressure (e.g., the burst pressure of the second stop valve) and the fourth burst pressure (e.g., the burst pressure of the fourth stop valve) are both less than the first burst pressure (e.g., the first burst pressure). Referring, for example, to FIG. 3, in some embodiments wherein the burst pressure of second stop valve 112 (e.g., the second burst pressure) and the burst pressure of fourth stop valve 134 (e.g., the fourth burst pressure) are the same, the burst pressure of second stop valve 112 and the burst pressure of fourth stop valve 134 may be less than the burst pressure of first stop valve 106 (e.g., the first burst pressure).

In certain embodiments, the microfluidic device may comprise a second flow path disposed between the control channel and the second reservoir. Referring, for example, to FIG. 3, microfluidic device 101b comprises second flow path 117 disposed between control channel 104 and second reservoir 130. Second flow path 117 may, in some embodiments, comprise a flow path through fourth stop valve 134. According to some embodiments, although not shown in the figures, the second flow path may comprise a flow path through one or more passive components (e.g., a channel) in addition to the fourth stop valve, as the disclosure is not meant to be limiting in this regard.

The first flow path and the second flow path may be separate from each other, in some embodiments. For example, as shown in FIG. 3, first flow path 115 and second flow path 117 are separate from each other. In some embodiments, the first flow path and the second flow path may be separate from each other such that no portion of the first flow path extending between the control channel and the first reservoir is coextensive with a portion of the second flow path extending between the control channel and the second reservoir. Referring to FIG. 3, for example, no portion of first flow path 115 extending between control channel 104 and first reservoir 108 is coextensive with a portion of second flow path 117 extending between control channel 104 and second reservoir 130.

The microfluidic device may comprise a stop valve fluidly connected to an outlet of the second reservoir. Referring to FIG. 3, for example, microfluidic device 101b comprises fifth stop valve 136 fluidly connected to outlet 138 of second reservoir 130. As shown in FIG. 3, outlet 138 is fluidly connected to a downstream portion (e.g., a most downstream portion) of reservoir 130, according to some embodiments.

The fifth stop valve may have a fifth burst pressure. In certain embodiments, the first burst pressure (e.g., the burst pressure of the first stop valve) is less than the third burst pressure (e.g., the burst pressure of the third stop valve) and the fifth burst pressure. Referring, for example, to FIG. 3, the burst pressure of first stop valve 106 (e.g., the first burst pressure) is less than the burst pressure of third stop valve 114 (e.g., the third burst pressure) and the burst pressure of fifth stop valve 136 (e.g., the fifth burst pressure). Configuring the device in this way advantageously allows a fluid to burst the first stop valve disposed along the control channel without bursting the third stop valve or the fifth stop valve.

The fifth burst pressure may be any of a variety of suitable pressures described above with respect to the third burst pressure, provided that the first burst pressure is less than the third burst pressure and the fifth burst pressure.

Although FIG. 3 only shows two reservoirs fluidly connected to the control channel at a position upstream from the first stop valve, a microfluidic device as described herein may comprise any appropriate number of reservoirs fluidly connected to the control channel and disposed upstream from the first stop valve, as the disclosure is not meant to be limiting this regarding. In some embodiments, for example, the microfluidic device may comprise three, four, five, or more reservoirs fluidly connected to the control channel at a position upstream from the first stop valve. Each reservoir disposed upstream from the first stop valve may be configured as described herein, for example, with a stop valve disposed between the control channel and the reservoir and a stop valve fluidly connected to an outlet of the reservoir. In some embodiments, each reservoir disposed upstream from the first stop valve may be configured as a series of cascading reservoirs such that the burst pressure of the second stop valve of the first reservoir is greater than the burst pressure of the second stop valve of the second reservoir, which is greater than the burst pressure of the second stop valve of the third reservoir, etc. In other embodiments, each reservoir disposed upstream form the first stop valve may be configured such that the burst pressure of the second stop valve of each reservoir is the same.

According to certain embodiments, a microfluidic device may comprise multiple stop valves disposed along the control channel. In some such embodiments, there may be any appropriate number of reservoirs, configured as described herein, positioned upstream from each stop valve disposed on the control channel.

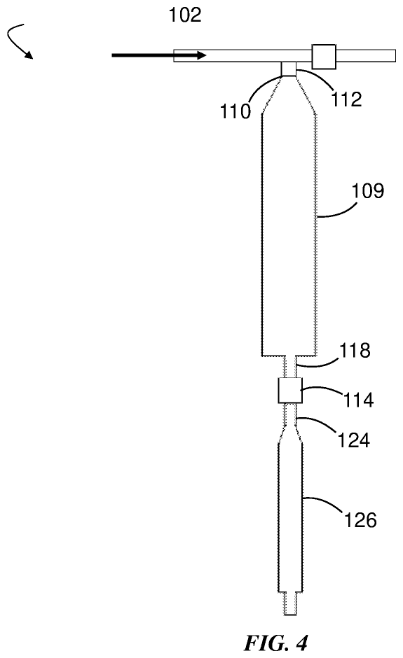

According to certain embodiments, a microfluidic device as described herein may be used to flow samples and/or reagents in one or more channels and/or reservoirs of the device. FIG. 4 shows, according to some embodiments, a schematic diagram of a microfluidic device comprising a control channel, a control reservoir, and a process reservoir. Referring to FIG. 4, microfluidic device 101c comprises control reservoir 109 (e.g., a first control reservoir). In certain embodiments, third stop valve 114 may be fluidly connected to inlet 124 of process reservoir 126. For example, in some embodiments, process reservoir 126 comprises inlet 124 fluidly connected to third stop valve 114. In some embodiments, the process reservoir may be configured to process one or more samples and/or reagents.

In some embodiments, a fluid (e.g., control fluid) flowing into microfluidic device 101c in direction 102 may burst second stop valve 112, thereby flowing into (and optionally filling) control reservoir 109 without bursting first stop valve 106 or third stop valve 114. As a result of the control fluid flowing into control reservoir 109, a positive pressure may be applied to process reservoir 126, thereby advantageously causing a process fluid (e.g., a sample fluid, a reagent fluid) positioned therein to flow. In some embodiments, for example, the fluid (e.g., air) displaced in control reservoir 109 by the control fluid may apply a positive pressure to process reservoir 126 through third stop valve 114 connected to outlet 118 of control reservoir 109. After reaching the appropriate threshold pressure, the control fluid in control reservoir 109 may burst first pressure valve 106 without bursting third stop valve 114. In some embodiments, the control fluid may be stored in control reservoir 109 without bursting third stop valve 114. The microfluidic device as configured in FIG. 4 may be used to route fluids (e.g., the control fluid, the process fluid) to different reservoirs on the device which advantageously provides the ability to perform time stepped sampling and/or aliquoting of defined volumes of the fluids.

According to certain embodiments, the control fluid may be any fluid that is capable of flowing through the microfluidic device. In some embodiments, the control fluid is used to provide a positive pressure in the control channel and to facilitate the flow of one or more process fluids in a process reservoir. In certain non-limiting embodiments, the control fluid may be water (e.g., deionized water) or a liquid buffer. In some embodiments, low surface tension liquids are not preferred as the control fluid since they can leak from the control channel due to their low contact angle, which can disturb the sequential liquid delivery process.

The control fluid may, in some embodiments, be stored in the control reservoir for later processing and/or sampling. In some non-limiting embodiments, for example, the control fluid is an effluent from an organ on chip (OOC) system, which may be stored in the control reservoirs and later analyzed, for example, with any appropriate spectroscopic and/or biosensing technique.

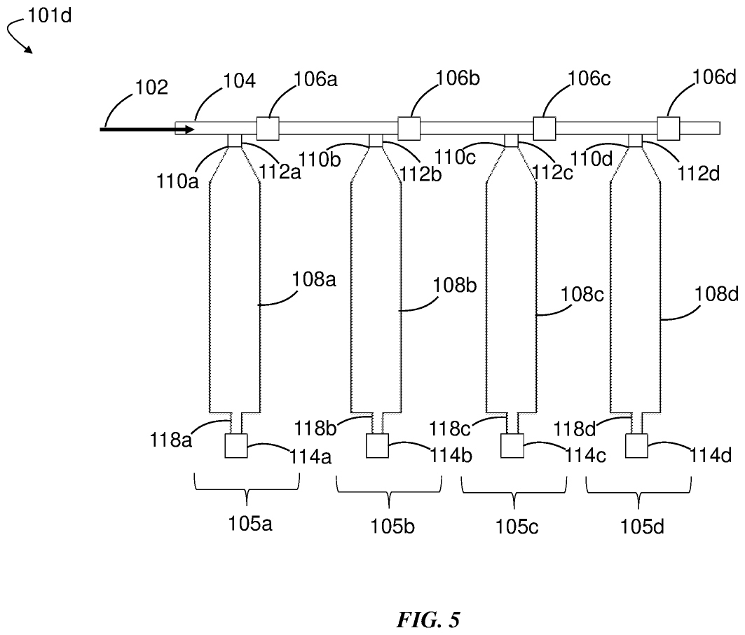

According to certain embodiments, a microfluidic device may comprise a plurality of cascading reservoirs disposed in series along at least a portion of a length of the control channel. FIG. 5 shows, according to some embodiments, a schematic diagram of microfluidic device 101d comprising control channel 104 and plurality of cascading reservoirs 105. In some embodiments, each cascading reservoir 105 includes a control channel stop valve (see, e.g., first stop valve 106), reservoir 108 with inlet 110 fluidly connected to control channel 104 and positioned upstream from the control channel stop valve, and an upstream reservoir stop valve (see, e.g., second stop valve 112) disposed between control channel 104 and inlet 110 of reservoir 108.

According to some embodiments, each control channel stop valve has a first burst pressure and each upstream reservoir stop valve has a second burst pressure, wherein each second burst pressure is less than each first burst pressure. Referring to FIG. 5, for example, the burst pressure of each upstream reservoir stop valve (e.g., second stop valve 112) is less than the burst pressure of each control channel stop valve (e.g., first stop valve 106). Configuring the microfluidic device in this way advantageously allows a fluid to burst the upstream reservoir stop valve of each cascading reservoir and autonomously flow into (and optionally fill) the reservoir without bursting the control channel stop valve. According to some embodiments, the resistance of each upstream reservoir stop valve (e.g., second stop valve 112) may be less than the resistance of each corresponding control channel stop valve (e.g., first stop valve 106).

In some embodiments, each cascading reservoir may comprise a downstream reservoir stop valve (see, e.g., third stop valve 114) fluidly connected to outlet 118 of reservoir 108. Each downstream reservoir stop valve may, in some embodiments, have a third burst pressure, wherein each first burst pressure is less than each third burst pressure. For example, referring to FIG. 5, the burst pressure of each control channel stop valve (e.g., first stop valve 106) is less than the burst pressure of each downstream reservoir stop valve (e.g., third stop valve 114). This configuration advantageously allows a fluid to burst the control channel stop valve of each cascading reservoir without bursting the downstream reservoir stop valve of each cascading reservoir, thereby allowing the fluid to flow along control channel 104 into the next cascading reservoir 105.

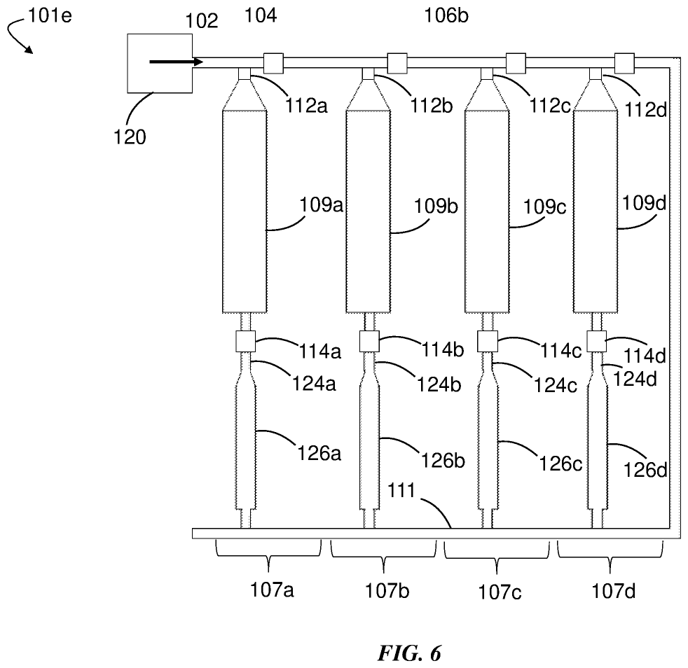

According to certain embodiments, each cascading reservoir may be a cascading control reservoir. FIG. 6 shows, according to some embodiments, a schematic diagram of microfluidic device 101e comprising inlet reservoir 120, control channel 104, and plurality of cascading control reservoirs 107. In some embodiments, each cascading control reservoir 107 comprises control reservoir 109, a control channel stop valve (see, e.g., first stop valve 106), an upstream reservoir stop valve (see, e.g., second stop valve 112), and a downstream reservoir stop valve (see, e.g., third stop valve 114). In some embodiments, microfluidic device 10le comprises plurality of process reservoirs 126, wherein each cascading control reservoir is fluidly coupled to one or more corresponding process reservoirs of the plurality of process reservoirs 126 through associated third stop valve 114. In some embodiments, for example, each process reservoir of the plurality of process reservoirs 126 has inlet 124 fluidly connected to each downstream stop valve 114.

As explained herein, a fluid (e.g., control fluid) flowing in control channel 104 may function as explained above with respect to FIG. 5, by, for example, bursting the upstream reservoir stop valve and filling the control reservoir without bursting the downstream reservoir stop valve. In some embodiments, the control fluid flowing in control reservoir 109 may displace a fluid (e.g., air) therein, thereby applying a positive pressure to the corresponding process reservoir 126 via the downstream stop valve. The application of a positive pressure from control reservoir 109 to process reservoir 126 causes a process fluid positioned in process reservoir 126 to flow. In some embodiments, the process fluid in each of process reservoirs 126 may flow to outlet channel 111. The process fluid may be, in certain embodiments, a sample fluid and/or a reagent fluid.

According to some embodiments, after filling control reservoir 109, the control fluid may burst the control channel stop valve upstream from the control reservoir without bursting the downstream reservoir stop valve. In certain embodiments, bursting the control channel stop valve causes the control fluid to cascade into the next cascading control reservoir, thereby bursting the subsequent upstream reservoir stop valve and flowing into the subsequent control reservoir, which causes a process fluid positioned in the corresponding process reservoir to flow. In some embodiments, the cascade configuration explained hercin may be repeated until the control reservoirs are filled.

According to some embodiments, the microfluidic device may comprise one or more passive components (e.g., channels, valves, reservoirs, other microfluidic components) that extend out of a plane relative to an adjacent component that the one or more passive components is fluidly connected to. In certain embodiments, for example, at least one dimension (e.g., a length, a width, and/or a depth) of a passive component (e.g., a stop valve) may extend out of a plane relative to an adjacent component that the passive component is fluidly connected to (e.g., a channel).

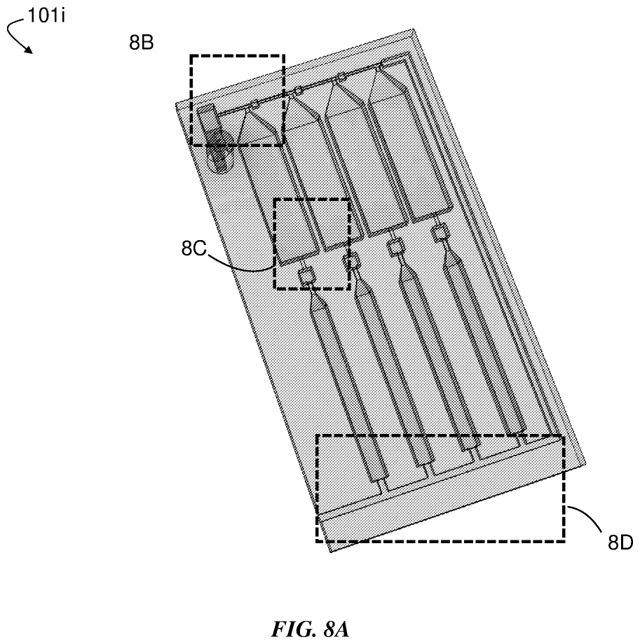

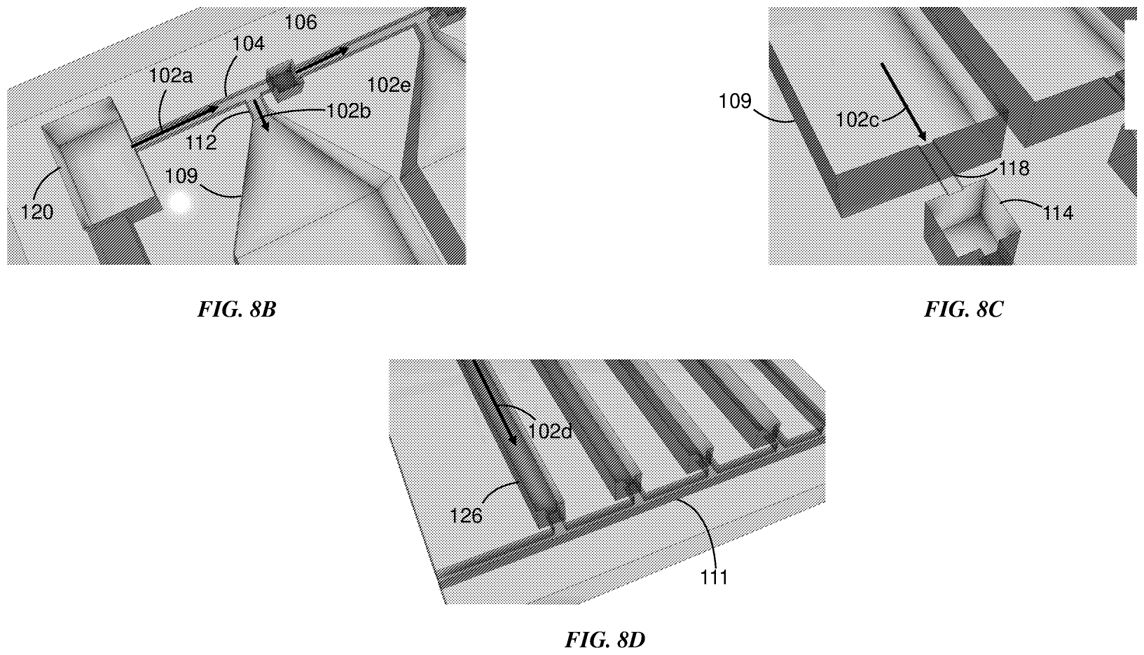

FIG. 8A shows, according to some embodiments, a schematic diagram of microfluidic device 101i comprising a plurality of passive components that extend out of a plane relative to an adjacent component that each passive component is fluidly connected to. FIG. 8B shows, according to some embodiments, a schematic diagram of passive components inlet reservoir 120, control channel 104, first stop valve 106, second stop valve 112, and control reservoir 109 of microfluidic device 101i, wherein inlet reservoir 120, first stop valve 106, and control reservoir 109 extend out of a plane relative to an adjacent component that each passive component is fluidly connected to. For example, as shown in FIG. 8B, inlet reservoir 120 extends out a plane relative to control channel 104, first stop valve 106 extends out of a plane relative to control channel 104, and control reservoir 109 extends out of a plane relative to second stop valve 112 and/or control channel 104. FIG. 8C shows, according to some embodiments, a schematic diagram of passive components control reservoir 109, outlet 118, and third stop valve 114 of microfluidic device shown 101i, wherein control reservoir 109 and third stop valve 114 extend out of a plane relative to an adjacent component that each passive component is fluidly connected to. For example, as shown in FIG. 8C, control reservoir 109 extends out of a plane relative to outlet 118, and third stop valve 114 extends out of a plane relative to outlet 118. FIG. 8D shows, according to some embodiments, a schematic diagram of passive components process reservoir 126 and outlet channel 111 of microfluidic device 101i, wherein process reservoir 126 extends out of a plane relative to an adjacent component that the passive component is fluidly connected to. For example, as shown in FIG. 8D, process reservoir 126 extends out of a plane relative to outlet channel 111. It should be understood that one or more other passive components of a microfluidic device not shown in FIGS. 8A-8D may also extend out of a plane relative to an adjacent component that the passive component is fluidly connected to, as the disclosure is not meant to be limiting in this regard.

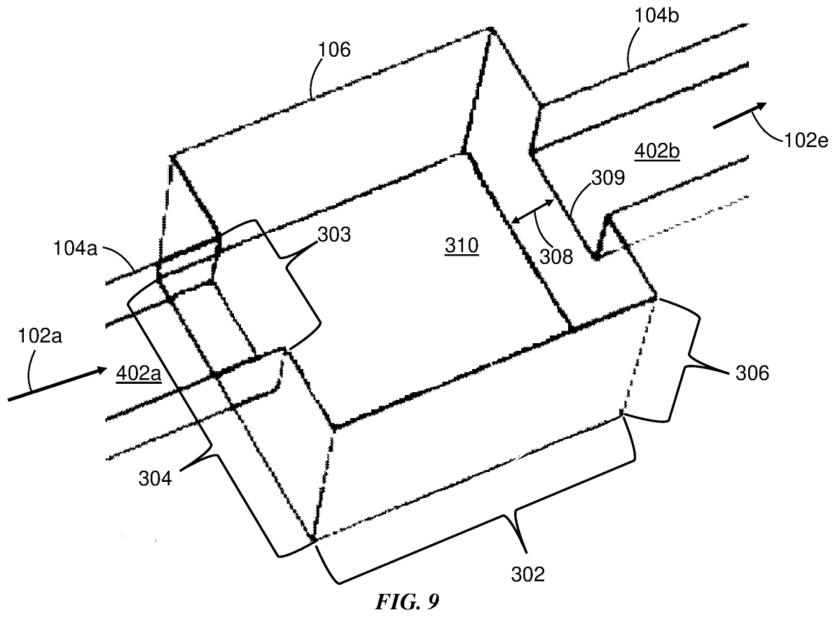

FIG. 9 shows, according to some embodiments, a schematic diagram of first stop valve 106 shown in FIG. 8B. As shown in FIG. 9, first stop valve 106 is disposed along control channel 104. In certain embodiments, first stop valve 106 has length 302, width 304, and depth 306. According to some embodiments, at least one dimension of the stop valve may extend out of a plane relative to the control channel. Referring to FIG. 9, for example, first stop valve 106 has depth 306 that extends out of plane 402 (e.g., upstream plane 402a and downstream plane 402b) of control channel 104 (e.g., upstream portion of control channel 104a and downstream portion of control channel 104b). In some embodiments, first stop valve 106 has out of plane depth 308 relative to plane 402 (e.g., upstream plane 402a and downstream plane 402b) of control channel 104 (e.g., upstream portion of control channel 104a and downstream portion of control channel 104b). Although FIG. 9 shows that depth 306 of first stop valve 106 is larger than out of plane depth 308 of first stop valve 106, in some embodiments the out of plane depth of the first stop valve may be the same as the depth of the first stop valve, as the disclosure is not meant to be limiting in this regard.

The length of a stop valve (e.g., a first stop valve, a second stop valve, a third stop valve, etc.) that extends out of a plane relative to an adjacent component that the stop valve is fluidly connected to may be any of a variety of suitable values. In some embodiments, the length of a stop valve that extends out of a plane relative to an adjacent component that the stop valve is fluidly connected to is greater than or equal to 1 micrometer, greater than or equal to 5 micrometers, greater than or equal to 10 micrometers, greater than or equal to 50 micrometers, greater than or equal to 0.1 mm, greater than or equal to 0.5 mm, greater than or equal to 1 mm, or greater than or equal to 5 mm. In some embodiments, the length of a stop valve that extends out of a plane relative to an adjacent component that the stop valve is fluidly connected to is less than or equal to 10 mm, less than or equal to 5 mm, less than or equal to 1 mm, less than or equal to 0.5 mm, less than or equal to 0.1 mm, less than or equal to 50 micrometers, less than or equal to 10 micrometers, or less than or equal to 5 micrometers. Combinations of the above recited ranges are possible (e.g., the length of a stop valve that extends out of a plane relative to an adjacent component that the stop valve is fluidly connected to is greater than or equal to 1 micrometer and less than or equal to 10 mm, the length of a stop valve that extends out of a plane relative to an adjacent component that the stop valve is fluidly connected to is greater than or equal to 0.1 mm and less than or equal to 0.5 mm). Other ranges are also possible.

The width of a stop valve (e.g., a first stop valve, a second stop valve, a third stop valve, etc.) that extends out of a plane relative to an adjacent component that the stop valve is fluidly connected to may be any of a variety of suitable values. In some embodiments, the width of a stop valve that extends out of a plane relative to an adjacent component that the stop valve is fluidly connected to is greater than or equal to 1 micrometer, greater than or equal to 5 micrometers, greater than or equal to 10 micrometers, greater than or equal to 50 micrometers, greater than or equal to 0.1 mm, greater than or equal to 0.5 mm, greater than or equal to 1 mm, or greater than or equal to 5 mm. In some embodiments, the width of a stop valve that extends out of a plane relative to an adjacent component that the stop valve is fluidly connected to is less than or equal to 10 mm, less than or equal to 5 mm, less than or equal to 1 mm, less than or equal to 0.5 mm, less than or equal to 0.1 mm, less than or equal to 50 micrometers, less than or equal to 10 micrometers, or less than or equal to 5 micrometers. Combinations of the above recited ranges are possible (e.g., the width of a stop valve that extends out of a plane relative to an adjacent component that the stop valve is fluidly connected to is greater than or equal to 1 micrometer and less than or equal to 10 mm, the width of a stop valve that extends out of a plane relative to an adjacent component that the stop valve is fluidly connected to is greater than or equal to 0.1 mm and less than or equal to 0.5 mm). Other ranges are also possible.

The depth of a stop valve (e.g., a first stop valve, a second stop valve, a third stop valve, etc.) that extends out of a plane relative to an adjacent component that the stop valve is fluidly connected to may be any of a variety of suitable values. In some embodiments, the depth of a stop valve that extends out of a plane relative to an adjacent component that the stop valve is fluidly connected to is greater than or equal to 1 micrometer, greater than or equal to 5 micrometers, greater than or equal to 10 micrometers, greater than or equal to 50 micrometers, greater than or equal to 0.1 mm, greater than or equal to 0.5 mm, greater than or equal to 1 mm, or greater than or equal to 5 mm. In some embodiments, the depth of a stop valve that extends out of a plane relative to an adjacent component that the stop valve is fluidly connected to is less than or equal to 10 mm, less than or equal to 5 mm, less than or equal to 1 mm, less than or equal to 0.5 mm, less than or equal to 0.1 mm, less than or equal to 50 micrometers, less than or equal to 10 micrometers, or less than or equal to 5 micrometers. Combinations of the above recited ranges are possible (e.g., the depth of a stop valve that extends out of a plane relative to an adjacent component that the stop valve is fluidly connected to is greater than or equal to 1 micrometer and less than or equal to 10 mm, the depth of a stop valve that extends out of a plane relative to an adjacent component that stop valve is fluidly connected to is greater than or equal to 0.1 mm and less than or equal to 0.5 mm). Other ranges are also possible.

As explained above, at least one dimension (e.g., a depth) of a stop valve (e.g., a first stop valve, a second stop valve, a third stop valve, etc.) may extend out of a plane relative to an adjacent component that the stop valve is fluidly connected to (e.g., a channel). An out of plane depth of a stop valve (e.g., a first stop valve, a second stop valve, a third stop valve, etc.) relative to an upstream portion and a downstream portion of a channel the stop valve is fluidly connected to may comprise any of a variety of suitable values. In some embodiments, the out of plane depth of a stop valve relative to an upstream portion and a downstream portion of a channel the stop valve is fluidly connected to is greater than or equal to 1 micrometer, greater than or equal to 5 micrometers, greater than or equal to 10 micrometers, greater than or equal to 50 micrometers, greater than or equal to 0.1 mm, greater than or equal to 0.5 mm, greater than or equal to 1 mm, or greater than or equal to 5 mm. In some embodiments, the out of plane depth of a stop valve relative to an upstream portion and a downstream portion of a channel the stop valve is fluidly connected is less than or equal to 10 mm, less than or equal to 5 mm, less than or equal to 1 mm, less than or equal to 0.5 mm, less than or equal to 0.1 mm, less than or equal to 50 micrometers, less than or equal to 10 micrometers, or less than or equal to 5 micrometers. Combinations of the above recited ranges are possible (e.g., the out of plane depth of a stop valve relative to an upstream portion and a downstream portion of a channel the stop valve is fluidly connected is greater than or equal to 1 micrometer and less than or equal to 10 mm, the out of plane depth of a stop valve relative to an upstream portion and a downstream portion of a channel the stop valve is fluidly connected is greater than or equal to 0.1 mm and less than or equal to 0.5 mm). Other ranges are also possible.

Referring to FIGS. 8B-8D, microfluidic device 101i is configured such that a fluid flows in direction 102a (e.g., from inlet reservoir 120) through control channel 104 to control reservoir 109. In some embodiments, the fluid bursts second stop valve 112 (e.g., when a pressure of the fluid is greater than a first pressure threshold corresponding to the burst pressure of second stop valve 112 and below a second pressure threshold corresponding to the burst pressure of first stop valve 106) and flows into control reservoir 109 via direction 102b. In some embodiments, the fluid may substantially fill control reservoir 109 via direction 102c. According to certain embodiments, as the fluid flows into control reservoir 109, a positive pressure may be applied to process reservoir 126, thereby causing a process fluid disposed therein to flow via direction 102d.

In certain embodiments, the first stop valve that extends out of a plane relative to the control channel is configured such that a fluid bursts the first stop valve when the pressure of the fluid is greater than the second pressure threshold corresponding to the burst pressure of the first stop valve. According to some embodiments, the out of plane dimension (e.g., out of plane depth) of the first stop valve may affect the burst pressure of the first stop valve. Referring to FIG. 9, for example, as the fluid flows in upstream portion of control channel 104a (e.g., via direction 102a), the fluid may enter first stop valve 106 and contact bottom surface 310 of first stop valve 106. In some embodiments, however, due the out of plane depth of the first stop valve, the pressure of the fluid may not be large enough to burst the first stop valve until the fluid fills the out of plane depth of the first stop valve. Referring to FIG. 9, for example, first stop valve 106 is configured such that the fluid bursts first stop valve 106 when the fluid fills out of plane depth 308 between bottom surface 310 and outlet 309 of first stop valve 106. In certain embodiments, once the fluid fills out of plane depth 308 between bottom surface 310 and outlet 309 of first stop valve 106, the pressure of the fluid is greater than the second pressure threshold corresponding to the burst pressure of first stop valve 106, and the fluid bursts first stop valve 106 and flows in downstream portion of control channel 104b (e.g., via direction 102c).

Although FIG. 9 shows that first stop valve 106 has a stepped configuration, i.e., a stepped change in height from bottom surface 310 of stop valve 106 to outlet 309 of stop valve 106, the first stop may be configured such that the first stop valve has a ramped configuration, i.e., a ramped changed in height from bottom surface 310 of stop valve 106 to outlet 309 of stop valve 106, as the disclosure is not meant to be limiting in this regard.

FIG. 9 shows and describes first stop valve 106 that extends out of a plane relative to control channel 104, but other stop valves (and other passive components) of the microfluidic devices described herein may extend out of a plane relative to an adjacent component that the passive component is fluidly connected to, and such passive components may function the same or similarly to first stop valve 106, as the disclosure is not meant to be limiting in this regard. In certain embodiments, for example, an inlet reservoir (e.g., inlet reservoir 120 shown in FIG. 8B), a control reservoir (e.g., control reservoir 109 shown in FIGS. 8B and 8C), a third stop valve (e.g., third stop valve 114 shown in FIG. 8C) and/or a process reservoir (e.g., process reservoir 126 shown in FIG. 8D) may extend out of a plane relative to an adjacent component that the passive component is fluidly connected to, and may function the same or similarly to first stop valve 106 shown and described in FIG. 9.

The passive components (e.g., channels, valves, reservoirs, other microfluidic components) extending out of a plane relative to an adjacent component that the passive components are fluidly connected to may be formed in the microfluidic device using any of the fabrication methods described herein. In some embodiments, a passive component extending out of a plane relative to an adjacent component that the passive component is fluidly connected to is fabricated using individual layers to form different portions of the passive component. According to certain embodiments, for example, a plurality (e.g., two or more) of patterned films may be used to fabricate the passive component, wherein each film has at least a portion of a profile of a desired portion of the passive component formed in the plurality of patterned films.

In other embodiments, a passive component extending out of a plane relative to an adjacent component that the passive component is fluidly connected to may be formed by additive manufacturing. Other methods of fabricating the passive components extending out of a plane relative to an adjacent component that the passive component is fluidly connected to, such as laser cutting, injection molding, hot embossing, machining, and the like, are also possible, as the disclosure is not meant to be limiting in this regard. In certain embodiments, regardless of the fabrication technique, the out of plane portion of the passive component (e.g., one or more surfaces of the out of plane portion of the passive component, such as a bottom surface and/or one or more side walls) may comprise one or more different materials and/or surface modifications relative to other portions of the passive component and/or the adjacent component that the passive component is fluidly connected to.

According to certain embodiments, the microfluidic device may comprise one or more passive components (e.g., channels, valves, reservoirs, other microfluidic components) that have at least one dimension that may be larger than a corresponding dimension of an adjacent component that the passive component is fluidly connected to. In some embodiments, for example, at least one dimension (e.g., a length, a width, and/or a depth) of a passive component (e.g., a stop valve) may be larger than a corresponding dimension of an adjacent component that the passive component is fluidly connected to (e.g., a channel). Referring to FIG. 9, for example, stop valve 106 has width 304 that is larger than width 303 of control channel 104 (e.g., upstream portion of control channel 104a and downstream portion of control channel 104b).

In some embodiments, the at least one dimension (e.g., width) of the stop valve may be greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 100% larger than the corresponding dimension (e.g., width) of the adjacent component that the stop valve is fluidly connected to (e.g., a channel). In certain embodiments, the at least one dimension (e.g., width) of the stop valve may be less than or equal to 150%, less than or equal to 100%, less than or equal to 90%, less than or equal to 80%, less than or equal to 70%, less than or equal to 60%, less than or equal to 50%, less than or equal to 40%, less than or equal to 30%, or less than or equal to 20% larger than the corresponding dimension (e.g., width) of the adjacent component that the stop valve is fluidly connected to (e.g., a channel). Combinations of the above recited ranges are possible (e.g., the at least one dimension of the stop valve may be greater than or equal to 10% and less than or equal to 150% larger than the corresponding dimension of the adjacent component that the stop valve is fluidly connected to, the at least one dimension of the stop valve may be greater than or equal to 50% and less than or equal to 60% larger than the corresponding dimension of the adjacent component that the stop valve is fluidly connected to). Other ranges are also possible.

According to some embodiments, the at least one dimension (e.g., width) of the first stop valve that is larger than the corresponding dimension (e.g., width) of the control channel may affect the burst pressure of the first stop valve. For example, referring to FIG. 9, as the fluid flows in upstream portion of control channel 104a (e.g., via direction 102a), the fluid may enter first stop valve 106 and contact bottom surface 310 of first stop valve 106. In some embodiments, however, due to width 304 of first stop valve 106 being larger than width 303 of control channel 104, the pressure of the fluid may not be large enough to burst the first stop valve until the fluid fills width 304 of first stop valve 106. In certain embodiments, once the fluid fills width 304 of first stop valve 106 (and, in some embodiments, fills out of plane depth 308, as explained above), the pressure of the fluid is greater than the second pressure threshold corresponding to the burst pressure of first stop valve 106, and the fluid bursts first stop valve 106 and flows into downstream portion of control channel 104b (e.g., via direction 102c).

FIG. 9 shows and describes first stop valve 106 that has at least one dimension that is larger than a corresponding dimension of control channel 104, but other stop valves (and other passive components) of the microfluidic device described herein may have at least one dimension that is larger than a corresponding dimension of an adjacent component that the passive component is fluidly connected to, and such passive components may function the same or similarly to first stop valve 106, as the disclosure is not meant to be limiting in this regard. In certain embodiments, for example, an inlet reservoir (e.g., inlet reservoir 120 shown in FIG. 8B), a control reservoir (e.g., control reservoir 109 shown in FIGS. 8B and 8C), a third stop valve (e.g., third stop valve 114 shown in FIG. 8C) and/or a process reservoir (e.g., process reservoir 126 shown in FIG. 8D) may have at least one dimension that is larger than a corresponding dimension of an adjacent component that the passive component is fluidly connected to, and may function the same or similarly to first stop valve 106 shown and described in FIG. 9.

The passive components (e.g., channels, valves, reservoirs, other microfluidic components) having at least one dimension larger than a corresponding dimension of an adjacent component that the passive components are fluidly connected to may be formed in the microfluidic device using any of the fabrication methods described herein. In some embodiments, a passive component having at least one dimension larger than a corresponding dimension of an adjacent component that the passive component is fluidly connected to is fabricated using individual layers to form different portions of the passive component. According to certain embodiments, for example, a plurality (e.g., two or more) of patterned films may be used to fabricate the passive component, wherein each film has at least a portion of a profile of a desired portion of the passive component formed in the plurality of patterned films. In other embodiments, a passive component having at least one dimension larger than a corresponding dimension of an adjacent component that the passive component is fluidly connected to may be formed by additive manufacturing. Other methods of fabricating the passive components having at least one dimension larger than a corresponding dimension of an adjacent component that the passive component is fluidly connected to, such as laser cutting, injection molding, hot embossing, machining, and the like, are also possible, as the disclosure is not meant to be limiting in this regard. In certain embodiments, regardless of the fabrication technique, at least a portion of the passive component (e.g., one or more surfaces of the passive component, such as a bottom surface and/or one or more side walls) may comprise one or more different materials and/or surface modifications relative to other portions of the passive component and/or the adjacent component that the passive component is fluidly connected to.

In certain embodiments, a sensor may be integrated into and/or otherwise associated with the microfluidic device to evaluate one or more fluids (e.g., a control fluid, a process fluid, etc.). In some embodiments, for example, a sensor may be integrated into one or more reservoirs (e.g., a control reservoir, a process reservoir) such that a fluid flowing in the reservoir may flow over and/or past the sensor. Suitable sensors include, but are not limited to, a chemical sensor (e.g., an electrochemical sensor), an optical sensor (e.g., UV-vis spectrometer, fluorescence spectrometer), and the like. The sensor may be used for the analysis of chemical or molecular components in the one or more fluids (e.g., a control fluid, a process fluid). Sensors integrated into and/or associated with a microfluidic device are explained herein in greater detail.

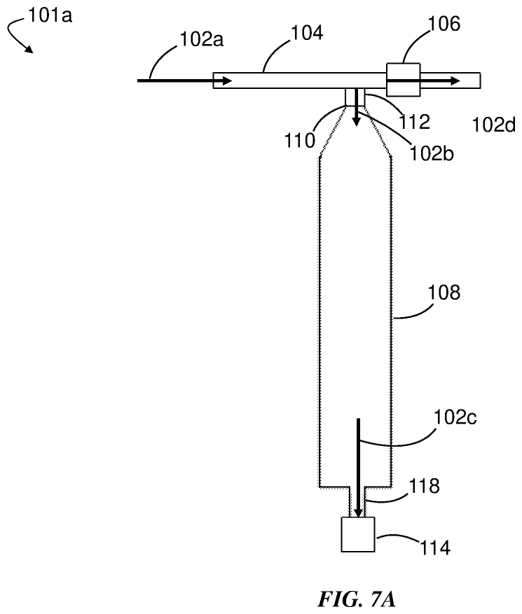

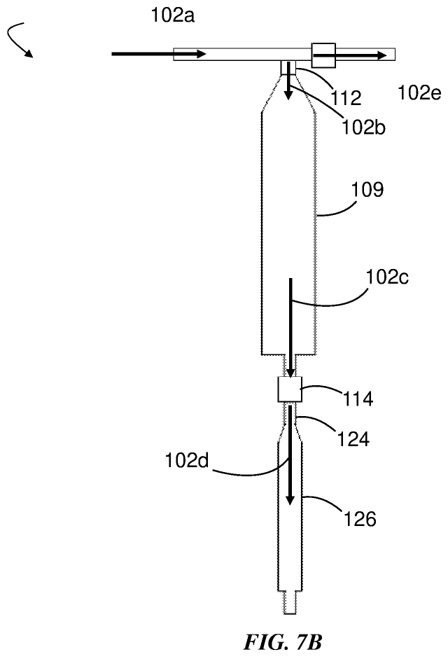

According to certain embodiments, a method of flowing a fluid in a microfluidic device is described. FIG. 7A shows, according to some embodiments, a schematic diagram of a microfluidic device comprising a control channel and a reservoir, depicting a method of flowing a fluid through the microfluidic device. In some embodiments, the method comprises flowing a fluid in direction 102a from a first portion of control channel 104 to reservoir 108. In certain embodiments, when a pressure of the fluid is greater than a first pressure threshold and below a second pressure threshold, the method comprises bursting second stop valve 112 and flowing the fluid into reservoir 108 via direction 102b. In some embodiments, for example, the first pressure threshold may correspond to the burst pressure of second stop valve 112 and the second pressure threshold may correspond to the burst pressure of first stop valve 106, such that the fluid has a pressure large enough to burst second stop valve 112 without bursting first stop valve 106. The method may, in some embodiments, comprise substantially filling reservoir 108 by flowing the fluid in reservoir 108 via direction 102c.

In certain embodiments, the method comprises flowing the fluid from the first portion of control channel 104 (e.g., upstream from first stop valve 106) to a second portion of control channel 104 (e.g., downstream from first stop valve 106) via direction 102d when the pressure of the fluid is greater than the second pressure threshold, thereby bursting first stop valve 106. In some embodiments, for example, the second pressure threshold corresponds to the burst pressure of first stop valve 106, such that the fluid has a pressure large enough to burst first stop valve 106 without bursting third stop valve 114.

According to certain embodiments and as described herein, more than one reservoir may be positioned upstream from first stop valve 106. In some such embodiments, the method may comprise flowing the fluid into a second reservoir prior to bursting the control channel stop valve (e.g., first stop valve 106). For example, in some embodiments wherein the microfluidic device comprises two reservoirs positioned upstream from the control channel stop valve, the fluid may fill the first reservoir as explained herein, and may have a pressure that is greater than the burst pressure of the upstream reservoir stop valve in the second reservoir but less than the second pressure threshold corresponding to the burst pressure of the control channel stop valve, thereby causing the fluid to flow into the second reservoir without bursting the control channel stop valve.

According to some embodiments in which the microfluidic device comprises a control channel, a control reservoir, and a process reservoir, the method may comprise flowing a process fluid in the process reservoir. FIG. 7B shows, according to some embodiments, a schematic diagram of a microfluidic device comprising a control channel, a control reservoir, and a process reservoir, depicting a method of flowing a control fluid and a process fluid in the microfluidic device. Referring to FIG. 7B, the method may comprise flowing a control fluid in control reservoir 109 as explained above with respect to FIG. 7A, and flowing a process fluid in process reservoir 126 via direction 102d as a result of flowing the first fluid from the first portion of control channel 104 (e.g., upstream from first stop valve 106) into control reservoir 109. In certain embodiments, flowing the control fluid into the control reservoir and flowing the process fluid in the process reservoir may occur simultaneously. According to some embodiments, and as explained herein in greater detail, the process fluid flows in process reservoir 126 due to the positive pressure applied from flowing the control fluid in control reservoir 109.

According to some embodiments, a system for testing an output from an organ on chip is described. In certain embodiments, the system comprises a microfluidic device (e.g., as described herein), a reservoir pod, and an organ on chip. In certain embodiments, the system may be configured such that the microfluidic device is configured to test the output from the organ on chip.

According to some embodiments, a reservoir pod is coupled to and/or otherwise associated with the organ on chip. In certain embodiments, for example, the reservoir pod comprises one or more fluid reservoirs that are configured to be associated with (e.g., fluidly connected to) the organ on chip such that a fluid inlet of the organ on chip is configured to receive one or more fluids from the one or more fluid reservoirs of the reservoir pod. In some embodiments, a fluid outlet of the organ on chip may be configured to be associated with (e.g., fluidly connected to) the microfluidic device such that microfluidic device is configured to receive the one or more fluids after the one or more fluids have flowed through the organ on chip. Configuring the system in this way advantageously allows a user to test an output from the organ on chip using the microfluidic device. In some embodiments, for example, the microfluidic device comprises one or more sensors (e.g., electrochemical sensors) that are configured to detect an analyte (e.g., biomarker) in the fluid after the fluid has flowed through the organ on chip. Additionally, the use of the microfluidic devices disclosed herein in such an arrangement may be advantageous. However, the use of other types of microfluidic devices with any desired functionality is also contemplated as the disclosure is not so limited.

The organ on chip may, in some embodiments, comprise a plurality of cells contained in at least one microfluidic channel and/or circuit that stimulates, replicates, and/or mimics the activities, mechanics, and/or physiological response of an organ or other biological structure. The organ on chip may be any of a variety of suitable organ on chips. In certain embodiments, for example, the organ on chip is a human lymphoid follicle-on-chip, a lung-on-chip, an intestine-on-chip, and/or a bone marrow-on-chip. Other organ on chips are also possible, however, as the disclosure is not meant to be limiting in this regard.

The human lymphoid follicle-on-chip is described in further detail in International Patent Application No. PCT/US2017/042657, filed Jul. 18, 2017, which is incorporated herein by reference in its entirety.

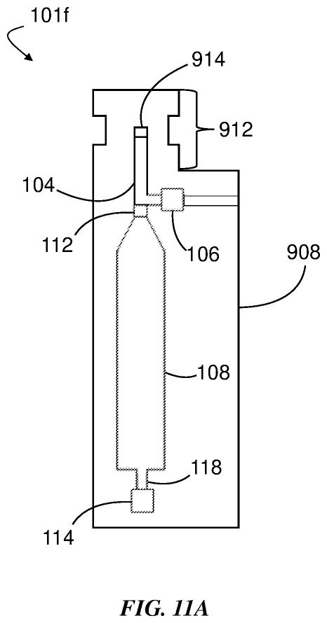

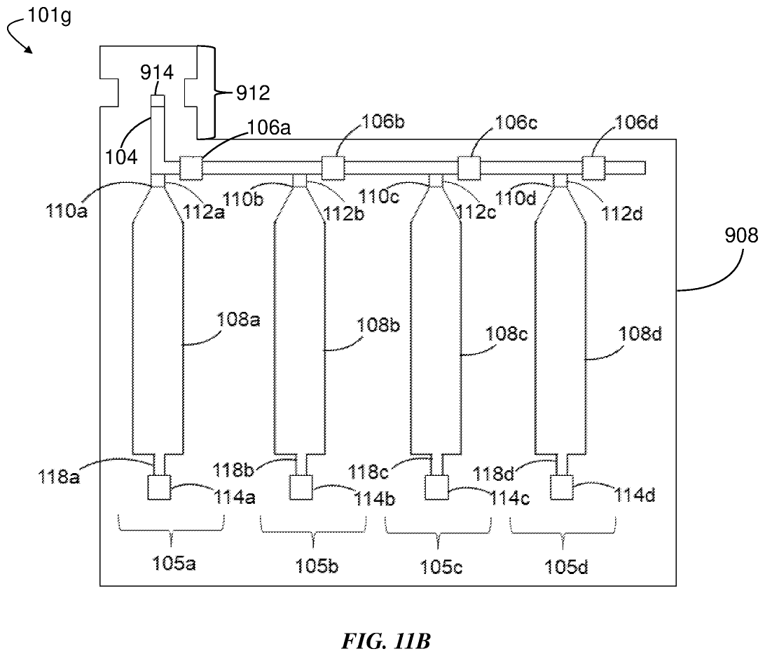

According to certain embodiments, the microfluidic device may be configured to test an output from an organ on chip. In some embodiments, the microfluidic device comprises a fluid inlet. FIG. 11A shows, according to some embodiments, a schematic diagram of microfluidic device 101f comprising fluid inlet 914. In certain embodiments, the fluid inlet may be fluidly connected to a control channel. Referring to FIG. 11A, for example, fluid inlet 914 is fluidly connected to control channel 104.

The fluid inlet of the microfluidic device is, in some embodiments, configured to be fluidly connected to a fluid outlet of an organ on chip. Referring to FIG. 11A, for example, fluid inlet 914 of microfluidic device 101f is configured to be fluidly connected to a fluid outlet of an organ on chip, for example, through an outlet port of the reservoir pod, as will be explained in greater detail herein.

The microfluidic device may, in some embodiments, comprise one or more reservoirs disposed at one or more locations along at least a portion of a length of the control channel. Referring to FIG. 11A, for example, microfluidic device 101f comprises reservoir 108 that is disposed along and is connected to control channel 104.

According to some embodiments, the microfluidic device may comprise any of a variety of suitable additional components (e.g., passive components) as described herein. In certain embodiments, for example, microfluidic device 101f may comprise first stop valve 106 (e.g., having a first burst pressure) disposed along control channel 104, second stop valve 112 (e.g., having a second burst pressure) disposed between control channel 104 and reservoir 108, and/or third stop valve 114 (e.g., having a third burst pressure) fluidly connected to outlet 118 of reservoir 108. In certain embodiments, microfluidic device 101f shown in FIG. 11A may function the same as or similarly to microfluidic device 101a shown in FIG. 1.

In certain embodiments, the microfluidic device comprises a plurality of reservoirs. FIG. 11B shows, according to some embodiments, a schematic diagram of a microfluidic device 101g comprising fluid inlet 914 and plurality of reservoirs 108. The plurality of reservoirs 108 may, in some embodiments, be disposed in series (e.g., sequentially) along at least a portion of a length of control channel 104. In certain embodiments, plurality of reservoirs 108 may be a plurality of cascading reservoirs (e.g., plurality of cascading reservoirs 105). According to some embodiments, microfluidic device 101g shown in FIG. 11B may function the same as or similarly to microfluidic device 101d shown in FIG. 5.

According to certain embodiments, the microfluidic device configured to test an output from an organ on chip may comprise a control channel, a control reservoir, and a process reservoir (e.g., similar to microfluidic device 101c shown in FIG. 4). In some embodiments, the microfluidic device configured to test an output from an organ on chip may comprise a control channel, a plurality of cascading control reservoirs, and a plurality of process reservoirs (e.g., similar to microfluidic device 101e shown in FIG. 6).

According to certain embodiments, the microfluidic device may comprise a body (e.g., a microfluidic device body). Referring to FIGS. 11A and 11B, for example, microfluidic devices 101f and 101g comprise microfluidic device body 908. The components (e.g., passive components) of the microfluidic device (e.g., channels, valves, reservoirs, other microfluidic components) may, in some embodiments, be formed in the microfluidic device body. As described herein in further detail, microfluidic device body 908 and/or the components (e.g., passive components) thereof (e.g., channels, valves, reservoirs, other microfluidic components) may be fabricated by additive manufacturing (e.g., 3D-printing).

In certain embodiments, the body (e.g., microfluidic device body) may comprise a connector configured to mate with a portion (e.g., one or more connection adapters) of the reservoir pod. Configuring the microfluidic device in this way advantageously allows the microfluidic device to be fluidly connected to the organ on chip associated with the reservoir pod. As shown in FIGS. 11A and 11B, for example, microfluidic devices 101f and 101g comprise connector 912 configured to mate with a portion (e.g., one or more connection adapters) of the reservoir pod. Suitable connections between the connector of the microfluidic device body and the portion (e.g., one or more connection adapters) of the reservoir pod are described herein in greater detail.

According to some embodiments, the microfluidic device comprises one or more sensors. The one or more sensors may, in some embodiments, be associated with the one or more reservoirs. Referring to FIG. 12, for example, microfluidic device 101h may comprise microfluidic device body 908 comprising one or more reservoirs (e.g., one or more reservoirs 108 as shown in FIGS. 11A and 11B), wherein one or more sensors 902 are associated with the one or more reservoirs. According to certain embodiments, each reservoir of the microfluidic device may be associated with one sensor. In other embodiments, each reservoir of the microfluidic device is associated with more than one sensor. In yet other embodiments wherein the microfluidic device comprises more than one reservoir, at least one reservoir may be associated with at least one sensor and at least one reservoir may not be associated with a sensor.

According to certain embodiments, the one or more sensors of the microfluidic device may comprise any appropriate type of sensor. For example, the one or more sensors may include one or more electrochemical sensors. Referring to FIG. 12, for example, one or more sensors 902 (e.g., sensors 902a, 902b, 902c, and/or 902d) may, in some embodiments, be chemical sensors (e.g., electrochemical sensors). Other sensors are also possible, however, as the disclosure is not meant to be limiting in this regard, including, for example, the use of optical sensors (e.g., UV-vis spectrometer, fluorescence spectrometer, and the like), field-effect transistor (FET) based biosensors, surface plasmon resonance (SPR) sensors, aptamer based sensors, and/or quartz crystal microbalance (QCM) sensors. In certain embodiments more than one type of sensor may be used with a microfluidic device. For example, both one or more chemical sensors (e.g., electrochemical sensors) and one or more optical sensors may be used. In certain embodiments, the one or more sensors may be one or more manual sensors and/or one or more automated sensors. According to some embodiments, for example, the one or more sensors may comprise and/or be associated with an automated microfluidic readout instrument that may be used to provide an analysis of the sensor (e.g., the detection of one or more analytes, such as one or more biomarkers).

In some embodiments, the one or more sensors (e.g., electrochemical sensors) are configured to detect one or more analytes. Any of a variety of suitable analytes may be detected by the one or more sensors. The one or more analytes may, in certain embodiments, comprise one or more biomarkers. In some embodiments, for example, the one or more biomarkers comprise cytokines; tissue inhibitors of metalloproteinases (TIMP), such as TIMP-1; matrix metalloproteinases (MMPs), such as MMP-1; macrophage inflammatory proteins (MIPs), such as MIP-1β; C-X-C motif chemokine ligand 13 (CXCL13), C-C motif chemokine ligand 5 (CCL5), interferon alpha (IFN-α), interleukin 6 (IL-6), and/or interleukin 8 (IL-8). Other analytes are also possible as the disclosure is not meant to be limiting in this regard.

In certain embodiments, the one or more sensors may be associated with the one or more reservoirs of the microfluidic device body via one or more sensor adhesives and/or one or more sealing films. Referring to FIG. 12, for example, one or more sensors 902 (e.g., sensors 902a, 902b, 902c, and 902d) may be associated with the one or more reservoirs of microfluidic device body 908 via sensor adhesives 904 (e.g., sensor adhesive 904a, 904b, 904c, and 904d) and sealing film 906a. In certain embodiments, one or more sensor adhesives 904 may be configured to adhere one or more sensors 902 to sealing film 906a. Scaling film 906a may, in certain embodiments, be configured to associate one or more sensors 902 adhered to sealing film 906a (e.g., via sensor adhesives 904) with one or more reservoirs of microfluidic device body 908.

Any of a variety of suitable sensor adhesives and/or sealing films may be employed. In certain embodiments, for example, the one or more sensor adhesives and/or sealing films may comprise a single sided adhesive tape, a double sided adhesive tape, an adhesive applied to the components to be bonded, and/or other appropriate types of adhesive arrangements. In some embodiments, the one or more sensor adhesives and/or sealing films comprise 3M™ Microfluidic Diagnostic Tape 9795R, 3M™ Microfluidic Diagnostic Film 9960, 3M™ Microfluidic Diagnostic Film 9962, 3M™ 9984 Diagnostic Microfluidic Surfactant Free Fluid Transport Film, ARclad® IS-8026 Silicone Transfer Adhesive, ARseal™ 90880 Polypropylene Double-Sided Adhesive Tape, ARflow® 93049 (Developmental) Hydrophilic Pressure Sensitive Adhesive, and/or Silicone Adhesive PET Tape. Other sensor adhesives and/or sealing films are also possible.

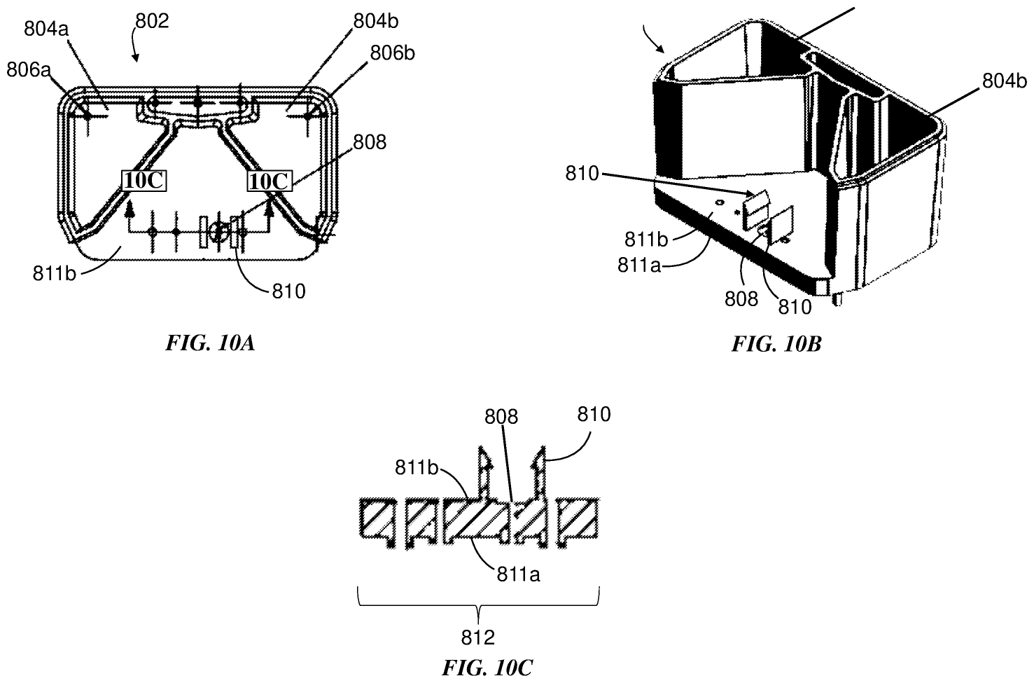

As noted above, in certain embodiments, a system for testing the output from the organ on chip comprises a reservoir pod. FIG. 10A shows one embodiment of a top-view schematic diagram of reservoir pod 802 and FIG. 10B shows one embodiment of a perspective view schematic diagram of reservoir pod 802.

The reservoir pod may, in some embodiments, comprise a fluid reservoir. In certain embodiments, the reservoir pod comprises more than one fluid reservoir (e.g., two fluid reservoirs, three fluid reservoirs, etc.). Referring to FIGS. 10A and 10B, for example, reservoir pod 802 comprises fluid reservoirs 804 (e.g., fluid reservoirs 804a and 804b).

According to certain embodiments, the reservoir pod comprises a fluid reservoir outlet. In some embodiments, the reservoir pod comprises more than one fluid reservoir outlet (e.g., two fluid reservoir outlets, three fluid reservoir outlets, etc.). The one or more fluid reservoir outlets may, in certain embodiments, be fluidly connected to the one or more fluid reservoirs. In some embodiments, for example, each fluid reservoir may comprise at least one fluid reservoir outlet. Referring to FIG. 10A, for example, reservoir pod 802 comprises fluid reservoir outlet 806a associated with fluid reservoir 804a and fluid reservoir outlet 806b associated with fluid reservoir 804b.

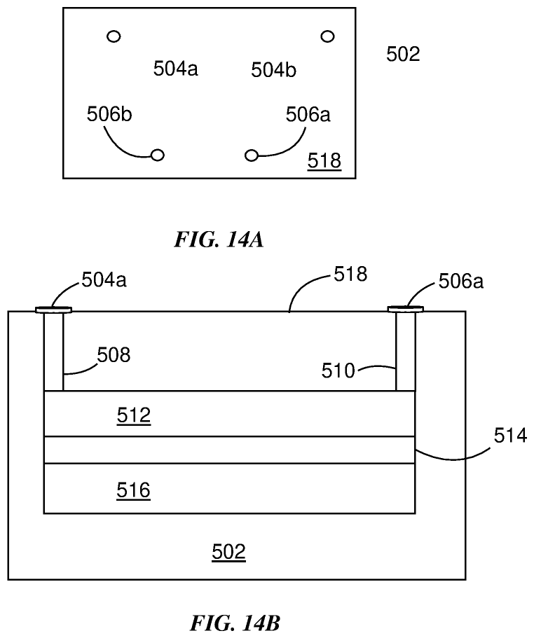

In certain embodiments, the fluid reservoir outlet is configured to be fluidly connected to a fluid inlet of an organ on chip. FIG. 14A shows, according to some embodiments, a top-view schematic diagram of organ on chip 502. Referring to FIGS. 10A and 14A, for example, fluid reservoir outlet 806 (e.g., reservoir outlet 806a) is, in some embodiments, configured to be fluidly connected, either directly or indirectly, to fluid inlet 504 (e.g., fluid inlet 504a) of organ on chip 502. In other embodiments, for example when the system is assembled, fluid reservoir outlet 806 (e.g., fluid reservoir outlet 806a) is fluidly connected to fluid inlet 504 (e.g., fluid inlet 504a) of organ on chip 502. As would be understood by a person of ordinary skill in the art, each fluid reservoir of the reservoir pod may be associated with one or more pressure sources (e.g., pressure pumps) configured to facilitate fluid flow from the fluid reservoir outlet to the fluid inlet of the organ on chip. Alternatively, in some embodiments a fluid reservoir of the reservoir pod may be pressurized by one or more passive flow inducing mechanisms. For example, in certain embodiments, hydrostatic pressure from the fluid in the fluid reservoir and/or capillary pressure may be used to draw fluid from the fluid reservoir into the organ on chip.

The fluid reservoir outlet is, in some embodiments, configured to flow a fluid from the fluid reservoir to the fluid inlet of the organ on chip. Referring to FIGS. 10A and 14A, for example, fluid reservoir outlet 806 (e.g., fluid reservoir outlet 806a) is configured to flow a fluid from fluid reservoir 804 (e.g., fluid reservoir 804a) to fluid inlet 504 (e.g., fluid inlet 504a) of organ on chip 502. In certain embodiments wherein the reservoir pod comprises more than one fluid reservoir outlet, each fluid reservoir outlet may be configured to be fluidly connected (or is fluidly connected, for example, when the system is assembled) to a fluid inlet of the organ on chip.

In certain embodiments, the reservoir pod comprises an outlet port. As shown in FIGS. 10A and 10B, for example, reservoir pod 802 comprises outlet port 808. Although only one outlet port is shown in the figures, the system may comprise more than one outlet port, as the disclosure is not meant to be limiting in this regard.

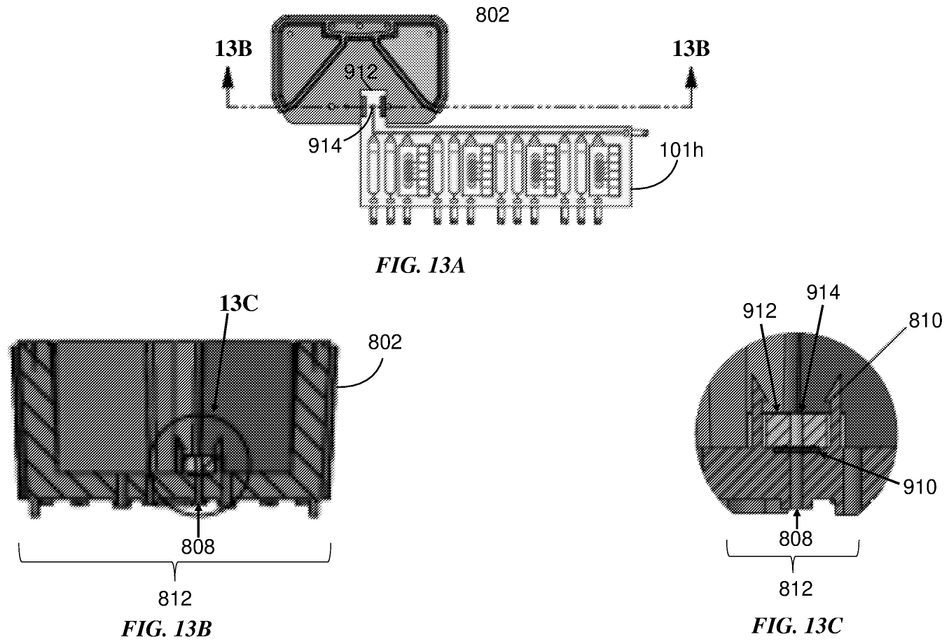

According to some embodiments, the outlet port is configured to be fluidly connected to the fluid inlet of the microfluidic device. In other embodiments, for example when the system is assembled, the outlet port is fluidly connected to the fluid inlet of the microfluidic device. According to some embodiments, FIG. 13A shows a top-view schematic diagram of a microfluidic device and a connected reservoir pod, FIG. 13B shows a cross-sectional schematic diagram of a microfluidic device and a connected reservoir pod taken along line 13B in FIG. 13A, and FIG. 13C shows a cross-sectional schematic diagram of a microfluidic device and a connected reservoir pod taken along circle 13C in FIG. 13B. As shown in FIGS. 13A-13C, outlet port 808 of reservoir pod 802 is fluidly connected to fluid inlet 914 of microfluidic device 101h.

FIG. 10C shows, according to certain embodiments, a cross-sectional schematic diagram of outlet port 808 taken along line 10C in FIG. 10A. The reservoir pod may, in some embodiments, comprise one or more connection adapters (e.g., one connection adapter, two connection adapters, three connection adapters, four connection adapters, etc.). As shown in FIGS. 10A-10C, for example, reservoir pod 802 comprises two connection adapters 810 such that outlet port 808 is disposed between the two connection adapters 810. The one or more connection adapters may, in some embodiments, comprise one or more locks and/or detents, although other connection adapters may also be employed as the disclosure is not meant to be limiting in this regard. In some embodiments, one or more connection adapters 810 may be configured to fluidly connect outlet port 808 to fluid inlet 114 of the microfluidic device. Referring to FIGS. 11A, 11B, and 13A, for example, microfluidic device 101 (e.g., microfluidic devices 101f, 101g, and 101h) may, in some embodiments, comprise connector 912 that is configured to mate with the connection adapter (e.g., connection adapter 810) such that the outlet port of the fluid reservoir (e.g., outlet port 808) is fluidly connected to fluid inlet 914 of microfluidic device 101.

Although one or more reservoir pod connection adapters and a microfluidic device connector are shown in the figures, the reservoir pod and the microfluidic device may be fluidly and physically connected with one another in any appropriate manner using any appropriate type of connector and/or connections. In some embodiments, for example, appropriate types of connecters may include a compression connector, male and female connectors, and/or any other appropriate type of connection that may form a fluid connection between an inlet and an outlet of the disclosed components. Additionally, in some embodiments, the one or more connections may be configured to maintain the one or more connections in a connected configuration using, for example, a lock. Accordingly, a connector may include detents, fasteners, mechanically interlocking parts, interference fits, adhesives, welds, and/or any other appropriate type of connection that maintains the reservoir pod and the microfluidic device in fluid communication with one another during operation.

According to certain embodiments, it may be desirable to provide a seal between the reservoir pod and the microfluidic device. For example, a gasket or other seal may be positioned at the interface between the outlet port and the fluid inlet of the microfluidic device. Referring to FIGS. 12 and 13C, for example, gasket 910 is positioned at the interface between outlet port 808 and fluid inlet 914 of the microfluidic device. In certain embodiments, the gasket advantageously permits no or substantially no fluid leakage between the outlet port and the fluid inlet of the microfluidic device.

In certain embodiments, the outlet port of the reservoir pod is configured to be fluidly connected to a fluid outlet of the organ on chip. Referring, for example, to FIGS. 10C and 14A, outlet port 808 is configured to be fluidly connected to fluid outlet 506 (e.g., fluid outlet 506a) of organ on chip 502. In other embodiments, for example when the system is assembled, outlet port 808 is fluidly connected to fluid outlet 506 (e.g., fluid outlet 506a) of organ on chip 502.

In some embodiments, the reservoir pod may be coupled to and/or otherwise be associated with the organ on chip. According to certain embodiments, for example, a surface of the reservoir pod may be configured to mate with a surface of the organ on chip such that the outlet port of the reservoir pod is configured to be fluidly connected to the fluid outlet of the organ on chip. Referring to FIGS. 10C and 14A, for example, surface 811a of reservoir pod 802 (e.g., opposite of surface 811b comprising outlet port 808) may, in some embodiments, be connected, either directly or indirectly, to surface 518 of organ on chip 502 such that outlet port 808 is fluidly connected to fluid outlet 506 (e.g., fluid outlet 506a) of organ on chip 502. Appropriate types of connections may include, but are not limited to, detents, fasteners, mechanically interlocking parts, interference fits, adhesives, welds, and/or any other appropriate type of connection.

In some embodiments, when the reservoir pod, the organ on chip, and the microfluidic device are fluidly connected with one another, the organ on chip may be fluidly connected to the microfluidic device through the reservoir pod. According to certain embodiments, for example, the outlet port of the reservoir pod is configured to flow a fluid from the fluid outlet of the organ on chip to the fluid inlet of the microfluidic device. In some embodiments, outlet port 808 is configured to be fluidly connected to both fluid inlet 914 of microfluidic device 101 (e.g., via one or more connection adapters 810 and connector 912) and fluid outlet 506 of organ on chip 502. In other embodiments, for example when the system is assembled, outlet port 808 is fluidly connected to both fluid inlet 914 of microfluidic device 101 (e.g., via one or more connection adapters 810 and connector 912) and fluid outlet 506 of organ on chip 502. Configuring the system in this way advantageously allows a fluid to flow from the fluid outlet of the organ on chip to the fluid inlet of the microfluidic device (e.g., through outlet port 808 of reservoir pod 802) such that the microfluidic device can test an output from the organ on chip.

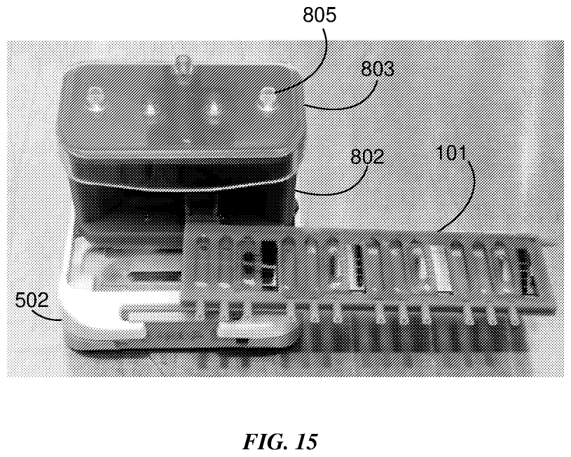

In certain embodiments, the reservoir pod may comprise a reservoir pod cover. FIG. 15 shows, according to some embodiments, an embodiment of a system comprising microfluidic device 101, reservoir pod 802, and organ on chip 502. As shown in FIG. 15, reservoir pod 802 may, in some embodiments, comprise reservoir pod cover 803. The reservoir pod cover may, in certain embodiments, advantageously protect one or more components of the reservoir pod (e.g., the one or more fluid reservoirs, the one or more fluid reservoir outlets, the outlet port, etc.) from, for example, cross contamination between fluid reservoirs and/or one or more external factors (e.g., liquids, debris, temperature fluctuations, etc.).

According to some embodiments, the reservoir pod cover may comprise one or more adapters that are configured to be connected to one or more pressure sources (e.g., pressure pumps). Referring to FIG. 15, for example, reservoir pod cover comprises adapter 805 that is configured to be connected to one or more pressure sources configured to facilitate fluid flow from the fluid reservoir outlet to the fluid inlet of the organ on chip. Of course, as described herein, fluid flow from the fluid reservoir to the organ on chip and to the microfluidic device may be facilitated by one or more passive flow inducing mechanisms (e.g., hydrostatic and/or capillary pressure), in some embodiments.

The reservoir pod and/or the reservoir pod cover may be fabricated by any of a variety of suitable techniques. In certain embodiments, at least a portion of the reservoir pod and/or the reservoir pod cover is fabricated by additive manufacturing (e.g., 3D-printing). In certain embodiments, the reservoir pod and/or the reservoir pod cover may be fabricated by any of a variety of suitable additive manufacturing processes, such as those described herein with respect to the microfluidic device (e.g., FDM, FFF, SLA, SLS, and the like). The reservoir pod and/or the reservoir pod cover may, in some embodiments, be fabricated using any of a variety of additive manufacturing materials, such as those described herein with respect to the microfluidic device (e.g., a thermoplastic, a thermosetting plastic, and/or a curable resin). However, the use of other appropriate manufacturing techniques such as hot embossing, injection molding, machining, etc., as well as other materials, may be used to form the reservoir pod, the reservoir pod cover, and/or any other component disclosed herein, as the disclosure is not limited to the described manufacturing techniques and/or materials used to form the various disclosed components.

According to certain embodiments, the system comprises an organ on chip. As described herein in further detail, the organ on chip may be coupled to and/or otherwise associated with the reservoir pod. In some embodiments, for example, the fluid reservoir outlet of the reservoir pod is configured to be fluidly connected to a fluid inlet of the organ on chip, and an outlet port of the reservoir pod is configured to be fluidly connected to a fluid outlet of the organ on chip. Configuring the device in this way advantageously allows a fluid to be flowed from the fluid reservoir of the reservoir pod, through the fluid reservoir outlet of the reservoir pod, through the fluid inlet of the organ on chip, through the organ on chip, through the fluid outlet of the organ on chip, through the outlet port of the reservoir pod, and to a fluid inlet of the microfluidic device configured to by fluidly connected to the outlet port of the reservoir pod.

FIG. 14B a cross-sectional schematic diagram of one embodiment of an organ on chip (e.g., a human lymphoid follicle-on-chip). The organ on chip may, in some embodiments, comprise a channel inlet fluidly connected to a fluid inlet of the organ on chip. Referring to FIG. 14B, for example, organ on chip 502 comprises channel inlet 508 fluidly connected to fluid inlet 504a. In certain embodiments, the organ on chip comprises a channel outlet fluidly connected to a fluid outlet of the organ on chip. As shown in FIG. 14B, for example, organ on chip 502 comprises channel outlet 510 fluidly connected to fluid outlet 506a.

Although not shown in the figures, the organ on chip may comprise additional channel inlets (e.g., a second channel inlet) fluidly connected to a fluid inlet (e.g., fluid inlet 504b) of the organ on chip and/or additional channel outlets (e.g., a second channel outlet) fluidly connected to a fluid outlet (e.g., fluid outlet 506b) of the organ on chip.

In some embodiments, the organ on chip comprises a first microchannel, a second microchannel, and a membrane (e.g., a semi-permeable membrane) disposed between the first microchannel and the second microchannel. Referring to FIG. 14B, for example, organ on chip 502 comprises first microchannel 512, second microchannel 516, and membrane 514 disposed between first microchannel 512 and second microchannel 516. Any of the channel inlets (e.g., channel inlet 508) and/or channel outlets (e.g., channel outlet 510) of organ on chip 502 may be fluidly connected to first microchannel 512 and/or second microchannel 516, according to some embodiments.

The organ on chip (e.g., human lymphoid follicle-on-chip) may, in some embodiments, include cells from human blood and lymphatic tissue and an extracellular matrix for the development of immune system components. According to certain embodiments, and as explained in further detail in International Patent Application No. PCT/US2017/042657, which is incorporated herein by reference in its entirety, the organ on chip (e.g., human lymphoid follicle-on-chip) may enable the study of lymphoid cells and/or lymphoid tissues in vitro, while mimicking at least some function of either of these cells and/or tissue or their responses to the stimuli and environment that cells and/or tissue are exposed to in vivo. Of course, other constructions of organ on chips as well as different types of cells and/or tissue may be used as the disclosure is not so limited.

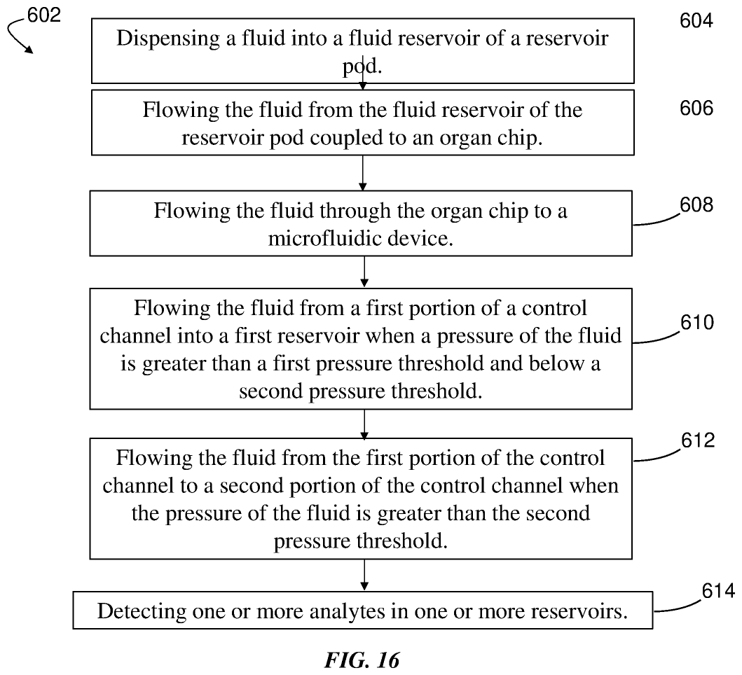

According to certain embodiments, a method of operating an organ on chip for analysis of an output (e.g., an effluent), or waste stream, from the organ on chip is described herein in regards to FIG. 16. The depicted embodiment describes a method 602 of a flowing a fluid through the system.

In certain embodiments, the method comprises step 604 comprising dispensing the fluid in a fluid reservoir of a reservoir pod. The fluid may be dispensed into the fluid reservoir by any of a variety of suitable means, including, for example, using pipettes (e.g., micropipettes).

In some embodiments, the method comprises step 606 comprising flowing the fluid from the fluid reservoir of the reservoir pod into an organ on chip. In certain embodiments, for example, step 606 comprises flowing the fluid from a fluid reservoir outlet of the fluid reservoir to a fluid inlet of an organ on chip fluidly connected to the fluid reservoir outlet.

According to some embodiments, the method comprises step 608 comprising flowing the fluid through the organ on chip to a microfluidic device. In some embodiments, for example, step 608 comprises flowing the fluid from the fluid inlet of the organ on chip to a fluid outlet of the organ on chip fluidly connected to an outlet port of the reservoir pod, and flowing the fluid from the fluid outlet of the organ on chip, through the outlet port of the reservoir pod, and to a fluid inlet of the microfluidic device fluidly connected to the outlet port of the reservoir pod.

As described herein in further detail, the microfluidic device may further comprise a control channel, one or more reservoirs disposed along at least a portion of a length of the control channel, a first stop valve (e.g., having a first burst pressure) disposed along the control channel, a second stop valve (e.g., having a second burst pressure) disposed between the control channel and the one or more reservoirs, and/or a third stop valve (e.g., having a third burst pressure) fluidly connected to an outlet of the one or more reservoirs. In some embodiments, the fluid inlet of the microfluidic device is fluidly connected to the control channel and upstream from the first stop valve and the second stop valve. Of course, it should be understood that other appropriate types of microfluidic devices may be fluidly connected to the reservoir pod and the organ on chip, as the disclosure is not limited to the specific microfluidic device used with the systems and methods disclosed herein.

According to certain embodiments, the method comprises step 610 comprising flowing the fluid from a first portion of the control channel into a first reservoir of the one or more reservoirs when a pressure of the fluid is greater than a first pressure threshold (e.g., corresponding to the burst pressure of the second stop valve) and below a second pressure threshold (e.g., corresponding to the burst pressure of the first stop valve).

In some embodiments, the method comprises step 612 comprising flowing the fluid from the first portion of the control channel to a second portion of the control channel when the pressure of the fluid is greater than the second pressure threshold (e.g., corresponding to the burst pressure of the first stop valve).

The microfluidic device may, in some embodiments, comprise one or more sensors (e.g., one or more electrochemical sensors) associated with the one or more reservoirs.

Thus, according to some embodiments, the method comprises step 612 comprising detecting one or more analytes (e.g., biomarkers) in the one or more reservoirs of the microfluidic device using, for example, the one or more sensors (e.g., one or more electrochemical sensors). Further, in instances in which the above noted microfluidic devices are used, this may result in sequential monitoring of one or more analytes (e.g., biomarkers) contained in the flow of liquid output from the organ on chip at separate sequential time points. This may advantageously allow for time-based monitoring of the organ on chip, in some embodiments.

The microfluidic devices described herein can be used, in some embodiments, for applications that require automated time stepped sampling and aliquoting of defined volumes of fluids. In some embodiments, for example, the plurality of cascading reservoirs can be used to aliquot fluids to discrete reservoirs by allowing the fluids to flow to each reservoir sequentially based on the differences in burst pressure of the stop valves without adding any additional connections or peripherals to the microfluidic device. The design of the microfluidic device therefore enables sequential delivery of fluids using a valving system in a compact and reliable manner. The microfluidic device can be used, for example, for biosensing applications.

### EXAMPLE 1

The following example describes the use of a microfluidic system comprising autonomous directional valves.

A microfluidic device comprising autonomous directional valves was used to sample a control fluid effluent from an organ on chip (OOC) system at fixed time intervals. The directional valves were used to direct the flow of the effluent to control reservoirs at different time points based on the structurally encoded flow sequence of the microfluidic device. The effluent was sampled and evaluated at different time points and stored in the control reservoirs. An electrochemical sensor and/or fluorescent probe were used to measure the levels of analytes and/or cytokines in the control fluid.

Flow of the control fluid in the control reservoirs applied a positive pressure in the process reservoirs connected to the outlets of the control reservoirs. This configuration employing control reservoirs and process reservoirs was used for sequential liquid delivery to perform biological assays for point of care diagnostics. First, as a result of the positive pressure applied by the flow of the control fluid in the control reservoir, a sample fluid flowed in the process reservoir over an electrochemical sensor and analytes in the sample fluid were captured by capture antibodies immobilized on the surface of the electrochemical sensor. Second, a wash fluid was flowed over the electrochemical sensor, followed next by a detection antibody step to form a sandwich assay on top of the electrodes of the electrochemical sensor. Next, an enzymatic amplification step was used to enhance the electrochemical signal. Lastly, an additional washing step was used to reduce nonspecific binding and the electrochemical signal was measured.

The microfluidic device was fabricated using various fabrication techniques such as hot embossing, injection molding, additive manufacturing (e.g., 3D-printing), or laser cutting of films. Different materials were used to fabricate the microfluidic device, including plastic polymers that were subjected to hot embossing or injection molding, additive manufacturing resins, hydrophobic films, and hydrophilic films.

### EXAMPLE 2

The following example describes the use of a system comprising a microfluidic device, a reservoir pod, and an organ on chip.

The microfluidic device, reservoir pod, and human lymphoid follicle-on-chip were assembled as described herein (as shown, for example, in FIG. 15).

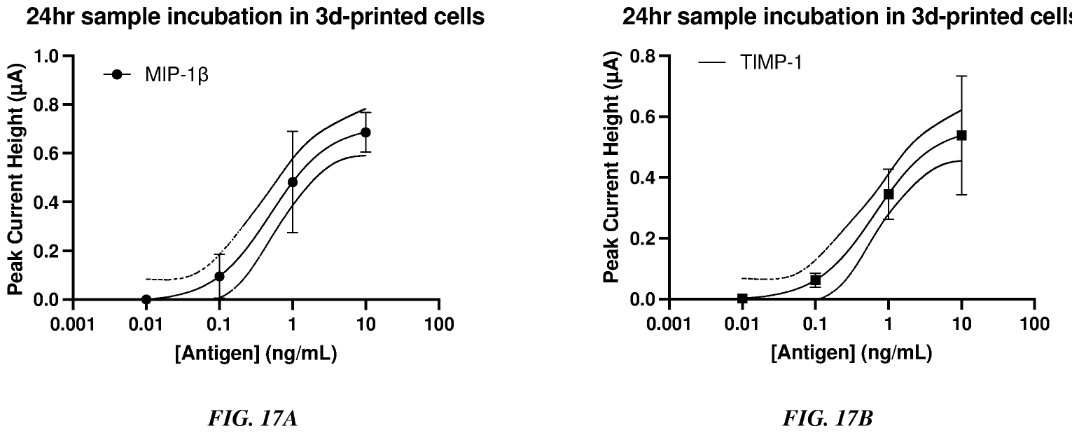

As shown in FIG. 17A, the binding curve for macrophage inflammatory protein MIP-1β was measured after incubating a 120 μl sample in a 3D-printed cell for 24 hours with an electrochemical sensor installed. As shown in FIG. 17B, the binding curve for tissue inhibitor of metalloproteinase TIMP-1 was also measured after incubating a 120 μl sample in a 3D-printed reservoir for 24 hours with the electrochemical sensor installed.

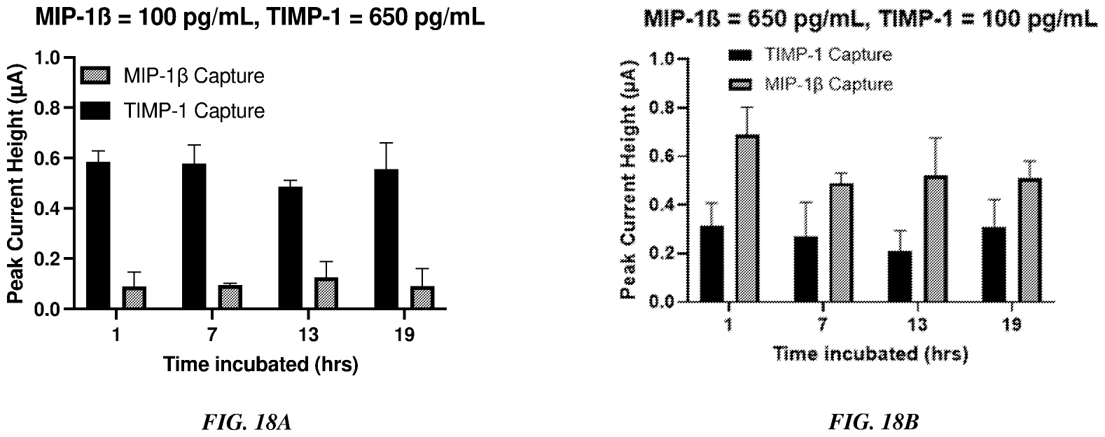

As shown in FIG. 18A, the electrochemical sensor simultaneously detected the concentration of the analytes from a sample incubated for 24 hours at 37° C., where a higher signal was observed for TIMP-1 and a lower signal was observed for MIP-1β, which corresponded to the actual concentration of the analytes present in the sample. As shown in FIG. 18B, the electrochemical sensor simultaneously detected the concentration of the analytes from a sample incubated for 24 hours at 37° C., where a higher signal was observed for MIP-1β and a lower signal was observed for TIMP-1, which corresponded to the actual concentration of the analytes present in the sample.

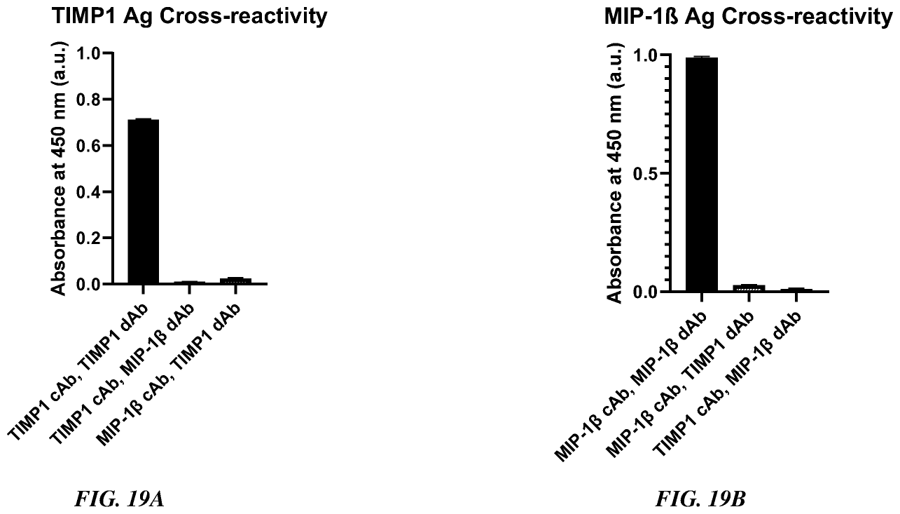

The cross reactivity on the surface of the electrochemical sensor was tested for TIMP-1 cAb against MIP-1β dAb and for MIP-1β cAb against TIMP-1 dAb, where a higher signal was only observed when the antibody pairs are matching (i.e., TIMP-1 cAb and TIMP-1 dAb as shown in FIG. 19A, and MIP-1β cAb and MIP-1β dAb as shown in FIG. 19B). The samples were incubated in a 3D-printed reservoir at 37° C. for 24 hours.

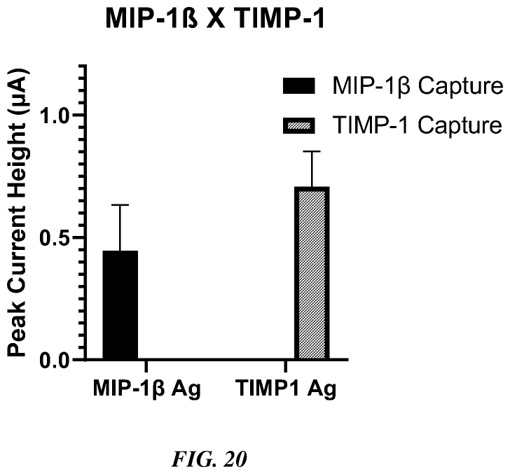

As shown in FIG. 20, MIP-1β and TIMP-1 were also simultaneously detected on the electrochemical sensor without cross reactivity.

While several embodiments have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple clements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A microfluidic device, comprising:
a control channel;
a first stop valve disposed along the control channel, wherein the first stop valve has a first burst pressure;
a first reservoir and a second reservoir fluidly connected to the control channel at a position upstream from the first stop valve;
a first flow path disposed between the control channel and the first reservoir, and a second flow path disposed between the control channel and the second reservoir, wherein the first flow path and the second flow path are separate from each other; and
a second stop valve disposed between the control channel and the first reservoir, wherein the second stop valve has a second burst pressure, and
wherein the second burst pressure is less than the first burst pressure.

2. The microfluidic device of claim 1, further comprising a third stop valve fluidly connected to an outlet of the first reservoir, and wherein the third stop valve has a third burst pressure.

3. The microfluidic device of claim 2, wherein the first burst pressure is less than the third burst pressure.

4. The microfluidic device of any one of claims 1-3, wherein the second stop valve is positioned at a fluidic interface between the control channel and the reservoir.

5. The microfluidic device of any one of claims 2-4, wherein the third stop valve is fluidly connected to a downstream portion of the first reservoir.

6. The microfluidic device of any one of claims 1-5, wherein the first reservoir is a first control reservoir.

7. The microfluidic device of claim 6, wherein the third stop valve is fluidly connected to an inlet of a process reservoir.

8. The microfluidic device of any one of claims 1-7, further comprising a fourth stop valve disposed between the control channel and the second reservoir, wherein the fourth stop valve has a fourth burst pressure.

9. The microfluidic device of claim 8, wherein the second burst pressure is less than the fourth burst pressure and the fourth burst pressure is less than the first burst pressure.

10. The microfluidic device of claim 8, wherein the second burst pressure is the same as the fourth burst pressure such that the second stop valve and the fourth stop valve are configured to burst simultaneously.

11. The microfluidic device of claim 10, wherein the second burst pressure and the fourth burst pressure are both less than the first burst pressure.

12. The microfluidic device of any one of claims 8-11, further comprising a fifth stop valve fluidly connected to an outlet of the second reservoir, wherein the fifth stop valve has a fifth burst pressure.

13. The microfluidic device of claim 12, wherein the first burst pressure is less than the third burst pressure and the fifth burst pressure.

14. The microfluidic device of any one of claims 1-13, wherein the microfluidic device comprises one or more intermediate layers that are configured to at least partially define a plurality of fluidly connected components.

15. The microfluidic device of claim 14, wherein the plurality of fluidly connected components includes at least the control channel, the first stop valve, the first reservoir, the second reservoir, and/or the second stop valve.

16. The microfluidic device of any one of claim 14-15, wherein the one or more intermediate layers are a plurality of intermediate layers.

17. The microfluidic device of any one of claims 1-16, further comprising a sensor integrated into or associated with the microfluidic device.

18. The microfluidic device of claim 17, wherein the sensor is an electrochemical sensor.

19. The microfluidic device of any one of claims 1-18, wherein at least a portion of the control channel, the first stop valve, the first reservoir, the second reservoir, and/or the second stop valve extend out of a plane relative to an adjacent component that the control channel, the first stop valve, the first reservoir, the second reservoir, and/or the second stop valve are fluidly connected to.

20. The microfluidic device of any one of claims 1-19, wherein at least a portion of the first stop valve extends out of a plane relative to an adjacent component that the first stop valve is fluidly connected to.

21. A microfluidic device, comprising:
a control channel;
a plurality of cascading control reservoirs disposed in series along at least a portion of a length of the control channel, wherein each cascading control reservoir includes:
a control reservoir with an inlet, wherein the inlet is fluidly connected to the control channel;
a first stop valve disposed along the control channel, wherein the inlet of the control reservoir is positioned upstream from the first stop valve;
a second stop valve disposed between the control channel and the inlet of the control reservoir; and
a third stop valve fluidly connected to an outlet of the reservoir, and
a plurality of process reservoirs, wherein each cascading control reservoir is fluidly coupled to one or more corresponding process reservoirs of the plurality of process reservoirs through the associated third stop valve, and wherein each cascading control reservoir is configured to apply a positive pressure to the one or more corresponding process reservoirs that causes a process fluid disposed in the one or more corresponding process reservoirs to flow.

22. The microfluidic device of claim 21, wherein each first stop valve has a first burst pressure and each second stop valve has a second burst pressure, wherein each second burst pressure is less than each first burst pressure.

23. The microfluidic device of any one of claims 21-22, wherein each third stop valve has a third burst pressure and each first burst pressure is less than each third burst pressure.

24. The microfluidic device of any one of claims 21-23, wherein the microfluidic device comprises one or more intermediate layers that are configured to at least partially define a plurality of fluidly connected components.

25. The microfluidic device of claim 24, wherein the plurality of fluidly connected components includes at least the control channel and the plurality of cascading control reservoirs.

26. The microfluidic device of any one of claims 24-25, wherein the one or more intermediate layers are a plurality of intermediate layers.

27. The microfluidic device of any one of claims 21-26, further comprising a sensor integrated into or associated with the microfluidic device.

28. The microfluidic device of claim 27, wherein the sensor is an electrochemical sensor.

29. The microfluidic device of any one of claims 21-28, wherein at least a portion of the control channel, the plurality of cascading control reservoirs, each first stop valve of the plurality of cascading reservoirs, each second stop valve of the plurality of cascading reservoirs, each third stop valve of the plurality of cascading reservoirs, and/or the plurality of process reservoirs extend out of a plane relative to an adjacent component that the control channel, the plurality of cascading control reservoirs, each first stop valve of the plurality of cascading reservoirs, each second stop valve of the plurality of cascading reservoirs, each third stop valve of the plurality of cascading reservoirs, and/or the plurality of process reservoirs are fluidly connected to.

30. The microfluidic device of any one of claims 21-29, wherein at least a portion of each first stop valve of the plurality of cascading control reservoirs extends out of a plane relative to an adjacent component that each first stop valve of the plurality of cascading control reservoirs is fluidly connected to.

31. A method of flowing a process fluid in a microfluidic device, comprising:
flowing a control fluid from a first portion of a control channel into a control reservoir when a pressure of the control fluid is greater than a first pressure threshold and below a second pressure threshold;
flowing the process fluid in a process reservoir fluidly connected to the control reservoir; and
flowing the control fluid from the first portion of the control channel to a second portion of the control channel when the pressure of the fluid is greater than the second pressure threshold.

32. The method of claim 31, wherein the microfluidic device comprises a first stop valve disposed along the control channel and a second stop valve disposed between the control channel and the control reservoir.

33. The method of any one of claims 31-32, wherein a burst pressure of the first stop valve corresponds to the second pressure threshold and a burst pressure of the second stop valve corresponds to the first pressure threshold.

34. The method of any one of claims 32-33, wherein flowing the control fluid from the first portion of the control channel into the control reservoir comprises bursting the second stop valve.

35. The method of any one of claims 32-34, wherein flowing the control fluid from the first portion of the control channel to the second portion of the control channel comprises bursting the first stop valve.

36. The method of any one of claims 31-35, wherein the microfluidic device further comprises a third stop valve fluidly connect to an outlet of the control reservoir.

37. The method of claim 36, wherein the third stop valve is fluidly connected to an inlet of the process reservoir.

38. The method of claim 37, wherein the process fluid flows in the process reservoir due to a positive pressure applied from the control reservoir.

39. A microfluidic device, comprising:
a control channel;
a first stop valve disposed along the control channel, wherein the first stop valve has a first burst pressure, and wherein the first stop valve extends out of a plane of the control channel;
a first reservoir fluidly connected to the control channel at a position upstream from the first stop valve; and
a second stop valve disposed between the control channel and the first reservoir, wherein the second stop valve has a second burst pressure, and
wherein the second burst pressure is less than the first burst pressure.

40. A microfluidic device, comprising:
a control channel;
a plurality of cascading control reservoirs disposed in series along at least a portion of a length of the control channel, wherein each cascading control reservoir includes:
a control reservoir with an inlet, wherein the inlet is fluidly connected to the control channel;
a first stop valve disposed along the control channel, wherein the inlet of the control reservoir is positioned upstream from the first stop valve, and wherein the first stop valve extends out of a plane of the control channel;
a second stop valve disposed between the control channel and the inlet of the control reservoir; and
a third stop valve fluidly connected to an outlet of the reservoir, and
a plurality of process reservoirs, wherein each cascading control reservoir is fluidly coupled to one or more corresponding process reservoirs of the plurality of process reservoirs through the associated third stop valve.

41. A system, comprising:
a microfluidic device;
a reservoir pod; and
an organ on chip,
wherein the reservoir pod comprises a fluid reservoir, a fluid reservoir outlet, and an outlet port,
wherein the fluid reservoir outlet is configured to be fluidly connected to a fluid inlet of the organ on chip, and
wherein the fluid inlet of the microfluidic device is configured to be fluidly connected to a fluid outlet of the organ on chip through the outlet port of the reservoir pod.

42. The system of claim 41, wherein the microfluidic device comprises a fluid inlet fluidly connected to a control channel, one or more reservoirs disposed along at least a portion of a length of the control channel, and one or more sensors associated with the one or more reservoirs.

43. The system of any one of claims 41-42, wherein the fluid reservoir outlet is fluidly connected to a fluid inlet of the organ on chip.

44. The system of any one of claim 41-43, wherein the fluid reservoir outlet is configured to flow a fluid from the fluid reservoir to the fluid inlet of the organ on chip.

45. The system of any one of claims 41-44, wherein the outlet port is configured to be fluidly connected to the fluid outlet of the organ on chip.

46. The system of any one of claims 41-45, wherein the outlet port is fluidly connected to the fluid outlet of the organ on chip.

47. The system of any one of claims 41-46, wherein the outlet port is configured to be fluidly connected to the fluid inlet of the microfluidic device.

48. The system of any one of claims 41-47, wherein the outlet port is fluidly connected to the fluid inlet of the microfluidic device.

49. The system of any one of claims 41-48, wherein the outlet port is configured to be fluidly connected to the fluid outlet of the organ on chip and the fluid inlet of the microfluidic device.

50. The system of any one of claims 41-49, wherein the outlet port is fluidly connected to the fluid outlet of the organ on chip and the fluid inlet of the microfluidic device.

51. The system of any one of claims 41-50, wherein the outlet port is configured to flow the fluid from the fluid outlet of the organ on chip to the fluid inlet of the microfluidic device.

52. The system of any one of claims 41-51, wherein the reservoir pod comprises one or more connection adapters configured to fluidly connect the outlet port to the fluid inlet of the microfluidic device.

53. The system of claim 52, wherein the microfluidic device comprises a body comprising a connector configured to mate with the one or more connection adaptors, wherein the connector comprises the fluid inlet.

54. The system of any one of claims 41-53, wherein a gasket is positioned between the outlet port and the fluid inlet of the microfluidic device.

55. The system of any one of claims 41-54, wherein the one or more sensors are associated with the one or more reservoirs via sealing film.

56. The system of any one of claims 41-55, wherein the one or more sensors comprise one or more electrochemical sensors.

57. The system of any one of claims 41-56, wherein the one or more sensors are configured to detect one or more biomarkers.

58. The system of any one of claims 41-57, wherein the organ on chip is a human lymphoid follicle-on-chip.

59. The system of any one of claims 41-58, wherein the microfluidic device comprises a plurality of reservoirs.

60. The system of claim 59, wherein the plurality of reservoirs is a plurality of cascading reservoirs disposed in series along at least the portion of the length of the control channel.

61. The system of claim 60, wherein the microfluidic device is the microfluidic device of any one of claims 21-30.

62. A method of flowing a fluid through a system, comprising:
flowing the fluid from a fluid reservoir of a reservoir pod into an organ on chip; and
flowing the fluid through the organ on chip to a microfluidic device.

63. The method of claim 62, wherein flowing the fluid from the fluid reservoir of the reservoir pod into the organ on chip comprises flowing the fluid from a fluid reservoir outlet of the reservoir pod to a fluid inlet of the organ on chip fluidly connected to the fluid reservoir outlet.

64. The method of claim 63, wherein flowing the fluid through the organ on chip to the microfluidic device comprises flowing the fluid from the fluid inlet of the organ on chip to a fluid outlet of the organ on chip fluidly connected to an outlet port of the reservoir pod.

65. The method of claim 64, wherein flowing the fluid through the organ on chip to the microfluidic device comprises flowing the fluid from the fluid outlet of the organ on chip, through the outlet port of the reservoir pod, and to a fluid inlet of the microfluidic device fluidly connected to the outlet port of the reservoir pod.

66. The method of claim 65, wherein the fluid inlet of the microfluidic device is fluidly connected to a control channel.

67. The method of claim 66, wherein the microfluidic device comprises one or more reservoirs disposed along at least a portion of a length of the control channel.

68. The method of any one of claim 67, further comprising:
flowing the fluid from a first portion of the control channel into a first reservoir of the one or more reservoirs when a pressure of the fluid is greater than a first pressure threshold and below a second pressure threshold; and
flowing the fluid from the first portion of the control channel to a second portion of the control channel when the pressure of the fluid is greater than the second pressure threshold.

69. The method of any one of claims 62-68, wherein the microfluidic device comprises one more sensors associated with the one or more reservoirs.

70. The method of claim 69, further comprising detecting one or more biomarkers in the one or more reservoirs.
